(19) United States
(12) Patent Application Publication
(10) Pub. No.: US 2022/0340971 A1
(43) Pub. Date: Oct. 27, 2022

(54) FRAGMENT ANALYSIS FOR QUANTITATIVE DIAGNOSTICS OF BIOLOGICAL TARGETS

(71) Applicant: BillionToOne, Inc., Menlo Park, CA (US)

(72) Inventors: Devon Brian Chandler Brown, Campbell, CA (US); Anna Bueno, Santa Clara, CA (US); David Tsao, San Carlos, CA (US); Oguzhan Atay, Menlo Park, CA (US)

(21) Appl. No.: 17/702,708

(22) Filed: Mar. 23, 2022

Related U.S. Application Data
(60) Provisional application No. 63/165,014, filed on Mar. 23, 2021.

(51) Int. Cl. C12Q 1/6883

(52) U.S. Cl. CPC C12Q 1/6883; C12Q 1/6858

(57) ABSTRACT

Aspects of the present disclosure include methods of detecting the presence or absence of one or more diseases using quantitative approaches. Aspects of the present disclosure include methods for determining the abundance of endogenous targets. Aspects of the present disclosure also include determining the presence or absence of an aneuploidy.

### CROSS REFERENCE TO RELATED APPLICATIONS

This application claims the benefit of U.S. Provisional application No. 63/165,014, filed Mar. 23, 2021, the disclosure of which is hereby incorporated in its entirety by reference.

### SEQUENCE LISTING

The instant application contains a Sequence Listing which has been submitted electronically in ASCII format and is hereby incorporated by reference in its entirety. Said ASCII copy, created on Apr. 18, 2022, is named 51812WO_CRF_sequencelisting.txt and is 11,230 bytes in size.

### BACKGROUND

Qualitative measurement of a nucleic acid may provide limited diagnostic information, for instance, in non-invasive prenatal testing. While quantitative testing does exist, it typically requires sequence-specific information. There exists a need for a cost-effective and fast method of quantitative analysis of nucleic acid species suitable for applications such as non-invasive prenatal testing (NIPT) and cancer detection, for a more accurate diagnosis of disorders.

### BRIEF DESCRIPTION OF THE DRAWINGS

FIG. 1 is a flowchart of a method of fragment analysis using spike-in molecules, according to one embodiment.

FIG. 2 is a flowchart of a method of aneuploidy detection, according to one embodiment.

FIG. 3 is a flowchart of an alternative method of aneuploidy detection, according to one embodiment.

FIG. 4 is a block diagram illustrating an example of aneuploidy detection, according to one embodiment.

FIG. 5 is a block diagram illustrating amplified genomic sequence molecules and spike-in molecules, according to one embodiment.

FIG. 6 is a flowchart of a method of single gene disorder detection, according to one embodiment.

FIG. 7 is a block diagram illustrating an example of sickle cell detection, according to one embodiment.

FIG. 8 is a block diagram illustrating an example of cystic fibrosis detection, according to one embodiment.

FIG. 9 is a is a block diagram illustrating an additional example of cystic fibrosis detection, according to one embodiment

FIG. 10 is a flowchart of an alternative method of single gene disorder detection, according to one embodiment.

FIG. 11 shows overall coefficient of variation (CV) results of capillary electrophoresis performed on cell free deoxyribonucleic acid (cfDNA) samples, according to one embodiment. Twelve injections and two spike-ins. (Negative: the cells were euploid, and the patient was pregnant). This is the equivalent of total noise.

FIG. 12 shows overall CV results of next generation sequencing (NGS) performed on the same sample as FIG. 11, according to one embodiment. Ratios were computed by summing reads to mimic capillary electrophoresis measurements. Ratios from both spike-ins were averaged. This removes measurement noise from the sample, but leaves “capture noise.”

FIG. 13 shows noise that is inherent in the sample, according to one embodiment. The two sources of noise are from the measurement and from the sample preparation (capture). Measurement noise was imputed by subtracting the capture noise from the total noise.

FIGS. 14 and 15 shows a decrease in noise relative to the number of reinjections, according to one embodiment. (Decreases percent CV by about half).

FIG. 16 shows measurement noise (without capture noise) for a single injection and for twelve injections, according to one embodiment.

FIGS. 17A-17B shows positive control gDNA samples containing the indicated fetal fraction of trisomy DNA (A: chromosome 18; B: chromosome 21) in a euploid background, according to one embodiment. Three replicates of each condition were tested.

FIG. 18 shows the experimental design for a respiratory panel design using qSanger to detect infectious diseases.

FIG. 19 shows the primers and genetic sequences for Influenza A, Influenza B, and SARS-CoV-2. Figure discloses SEQ ID NOS 1-15, respectively, in order of appearance.

FIGS. 20, 21, 22 and 23 show the spike-ins and experimental design for fragment analysis for sickle cell (HbS) single-gene non-invasive prenatal test (sgNIPT). FIG. 22 discloses SEQ ID NOS 16-17, 40, 20, 41, and 18-19, and FIG. 23 discloses SEQ ID NOS 16 and 21-23, respectively, in order of appearance.

FIGS. 24, 25, and 26 show the spike-ins and experimental design for fragment analysis for cystic fibrosis (F508del) sgNIPT. FIG. 26 discloses SEQ ID NOS 24-29 and 42-43, respectively, in order of appearance.

FIGS. 27, 28, 29, and 30 show an alternate spike-ins and experimental design for fragment analysis for cystic fibrosis (F508del) sgNIPT. FIG. 29 discloses SEQ ID NOS 30-31, 34, 32, 45, 33, 35, 44, and 46, and FIG. 30 discloses SEQ ID NOS 30 and 36-39, respectively, in order of appearance.

FIG. 31 shows a general experimental design for fragment analysis for infectious diseases.

### SUMMARY

Aspects of the present disclosure include methods of detecting the presence or absence of one or more diseases using quantitative approaches. Aspects of the present disclosure include methods for determining the abundance of endogenous targets. Aspects of the present disclosure also include determining the presence or absence of an aneuploidy.

Aspects of the present disclosure include a method of determining the presence or absence of an aneuploidy, the method comprising: mixing a DNA sample of a subject and a plurality of spike-in molecules to create a mixture, each of the plurality of spike-in molecules associated with a chromosome of a set of chromosomes, wherein each of the plurality of spike-in molecules comprises: a target region having a first nucleotide sequence with sequence similarity to a target sequence region of the respective chromosome, a variation region having a second nucleotide sequence with sequence dissimilarity to a sequence region of the respective chromosome, and co-amplifying the mixture with one or more chromosome-specific primers to create a co-amplified mixture; labeling the co-amplified mixture by chromosome with fluorescently labeled primers; receiving peak data from the co-amplified mixture, the peak data including, for each chromosome of the set of chromosomes, genomic peak intensities of the DNA sample and spike-in peak intensities of the spike-in molecules associated with the respective chromosome; for each chromosome, computing a ratio between the respective genomic peak intensity and the respective spike-in peak intensity; determining the presence or absence of the aneuploidy based on the computed ratios.

In some embodiments, the one or more chromosome-specific primers includes a set of chromosome-specific primers, each chromosome-specific primer in the set configured to capture a respective chromosome with a tail of a discrete length of a set of discrete lengths.

In some embodiments, computing, for each chromosome, the ratio between the respective genomic peak intensity and the respective spike-in peak intensity comprises: computing, for each discrete length of the set of discrete lengths, a ratio between the respective genomic peak intensity and the spike-in peak intensity; aggregating of the computed ratios across each discrete length of the set of discrete lengths.

In some embodiments, computing, for each chromosome, the ratio between the respective genomic peak and the respective spike-in peak intensity comprises: aggregating the genomic peak intensities across each discrete length of the set of discrete lengths; aggregating the spike-in peak intensities across each discrete length of the set of discrete lengths; computing a ratio between the aggregated genomic peak intensity and the aggregated spike-in peak intensity.

In some embodiments, the variation region includes an insertion of base pairs with a length of: one base pair, two base pairs, three base pairs, four base pairs, five base pairs, six base pairs, seven base pairs, eight base pairs, nine base pairs, ten base pairs, eleven base pairs, twelve base pairs, thirteen base pairs, fourteen base pairs, fifteen base pairs, sixteen base pairs, seventeen base pairs, eighteen base pairs, nineteen base pairs, or twenty base pairs.

In some embodiments, the variation region includes a deletion of base pairs with a length of: one base pair, two base pairs, three base pairs, four base pairs, five base pairs, six base pairs, seven base pairs, eight base pairs, nine base pairs, ten base pairs, eleven base pairs, twelve base pairs, thirteen base pairs, fourteen base pairs, fifteen base pairs, sixteen base pairs, seventeen base pairs, eighteen base pairs, nineteen base pairs, or twenty base pairs.

In some embodiments, a location of a respective variation region of a spike-in molecule is in the center of a respective amplicon of the spike-in molecule.

In some embodiments, each of the one or more fluorescently labeled primers is associated with a color channel.

Aspects of the present disclosure include a method of determining the presence or absence of an aneuploidy, the method comprising: for each chromosome in a set of chromosomes:mixing a DNA sample of a subject and a spike-in molecule of a plurality of spike-in molecules to create a mixture, each of the plurality of spike-in molecules associated with the chromosome of the set of chromosomes, wherein each of the plurality of spike-in molecules comprises: a target region having a first nucleotide sequence with sequence similarity to a target sequence region of the respective chromosome, a variation region having a second nucleotide sequence with sequence dissimilarity to a sequence region of the respective chromosome, co-amplifying the mixture with one or more primers of a set of primers to generate a co-amplified mixture, each primer configured to capture the respective chromosome and add a tail with a discrete length of a set of discrete lengths to an amplicon of the DNA sample and add a tail with the discrete length of the set of discrete lengths to an amplicon of the spike-in molecule; labeling the co-amplified mixture with fluorescently labeled primers; receiving peak data from the co-amplified mixture, the peak data including genomic peak intensities of the portion of the DNA sample for each discrete length of the set of discrete lengths and the spike-in peak intensities of the spike-in molecule for each discrete length of the set of discrete lengths; for each respective discrete length, computing a discrete length-specific ratio between the respective genomic peak intensity and the spike-in peak intensity; and aggregating the discrete length-specific ratios across each of the discrete lengths in the set of discrete lengths to generate a chromosome-specific ratio; and determining the presence or absence of aneuploidy based on the computed chromosome-specific ratios.

In some embodiments, determining the presence or absence of an aneuploidy based on the computed chromosome-specific ratios comprises: computing the ratio of a chromosome-specific ratio to each of the other chromosome-specific ratios; in response to determining a computed ratio is greater than a threshold ratio, determining the presence of aneuploidy; and in response to determining a computed ratio is less than a threshold ratio, determining the absence of aneuploidy.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

Aspects of the present disclosure include a method of determining the presence or absence of a genetic disorder in a noninvasive prenatal test, the method comprising: mixing a genomic sample of a subject and one or more spike-in molecules associated with the genetic disorder, each spike-in molecule associated with an allele of the genetic disorder, wherein the spike-in molecule comprises: a target region having a first nucleotide sequence with sequence similarity to a target sequence region of the respective allele of the genetic disorder, a variation region having a second nucleotide sequence with sequence dissimilarity to a sequence region of the respective allele of the genetic disorder, co-amplifying the mixture with one or more fluorescently labeled primers to generate a co-amplified mixture, wherein each of the one or more fluorescently labeled primers captures a respective allele of the genetic disorder, and wherein each of the fluorescently labeled primers generates an amplicon of the allele with a discrete length; receiving peak data from the co-amplified mixture, the peak data including, for each of the captured alleles, genomic peak intensities of the genomic sample and spike-in peak intensities of the spike-in molecules; computing, for each of the captured alleles, a ratio of the genomic peak intensity and the spike-in peak intensity; and determining the presence or absence of the genetic disorder based on a comparison of the computed ratios across each of the captured alleles.

In some embodiments, each of the captured alleles is associated with a color channel.

In some embodiments, an amplicon of a first allele of the captured alleles has a first length, an amplicon of a second allele of the captured alleles as a second length, and wherein the first length is shorter than the second length.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

In some embodiments, the genetic disorder is sickle cell.

In some embodiments, wherein a first spike-in molecule is associated with HbS allele, and wherein a second spike-in molecule is associated with HbA allele.

In some embodiments, computing the ratio for each of captured alleles comprises: computing a first ratio of peak intensities, wherein the first ratio is the ratio of the genomic peak intensity of the HbS allele and the spike-in intensity of the first spike-in molecule; computing a second ratio of peak intensities, wherein the second ratio is the ratio of the genomic peak intensity of the HbA allele and the spike-in intensity of the second spike-in molecule; and wherein determining the presence or absence a genetic disorder comprises determining the presence or absence of sickle cell disease based on a comparison of the first ratio and the second ratio.

In some embodiments, the genetic disorder is cystic fibrosis.

In some embodiments, a first spike-in molecule is associated with WT allele, and wherein a second spike-in molecule is associated with F508del allele.

In some embodiments, computing the ratio for each of captured alleles comprises: computing a first ratio of peak intensities, wherein the first ratio is the ratio of the genomic peak intensity of the WT allele and the spike-in intensity of the first spike-in molecule; computing a second ratio of peak intensities, wherein the second ratio is the ratio of the genomic peak intensity of the F508del allele and the spike-in intensity of the second spike-in molecule; and wherein determining the presence or absence a genetic disorder comprises determining the presence or absence of cystic fibrosis disease based on a comparison of the first ratio and the second ratio.

In some embodiments, each of the one or more fluorescently labeled primers is associated with a color channel.

Aspects of the present disclosure include a method of determining the presence or absence of a genetic disorder in a noninvasive prenatal test, the method comprising: mixing a genomic sample of a subject and a spike-in molecule associated with an allele of the genetic disorder to create a mixture, wherein the spike-in molecule includes a spike-in sequence, wherein the spike-in sequence comprises: a target region having a nucleotide sequence with sequence similarity to a target sequence region of the allele of the genetic disorder, a variation region having a nucleotide sequence with sequence dissimilarity to a sequence region of the allele of the genetic disorder, co-amplifying the mixture with one or more sets of allele-specific primers to generate a co-amplified mixture, each primer in a set of allele-specific primers configured to capture the respective allele and add a tail with a discrete length of a set of discrete lengths to an amplicon of the genomic sample and add a tail with the discrete length of the set of discrete lengths to an amplicon of the spike-in molecule, the amplicon of the genomic sample including the target sequence, the amplicon of the spike-in molecule including the spike-in sequence; labeling the co-amplified mixture with fluorescently labeled primers; receiving peak data from the co-amplified mixture, the peak data including for each discrete length of the set of discrete lengths, genomic peak intensities of the genomic sample and spike-in peak intensities of the spike-in molecule; for each respective discrete length, computing a ratio between the respective genomic peak intensity and the spike-in peak intensity; and determining the presence or absence of the genetic disorder based on the computed ratios.

In some embodiments, computing, for each allele, the ratio between the respective genomic peak intensity and the respective spike-in peak intensity comprises: computing, for each discrete length of the set of discrete lengths, a ratio between the respective genomic peak intensity and the spike-in peak intensity; aggregating of the computed ratios across each discrete length of the set of discrete lengths.

In some embodiments, computing, for each allele, the ratio between the respective genomic peak intensity and the respective spike-in peak intensity comprises: aggregating the genomic peak intensities across each discrete length of the set of discrete lengths; aggregating the spike-in peak intensities across each discrete length of the set of discrete lengths; computing a ratio between the aggregated genomic peak intensity and the aggregated spike-in peak intensity.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

In some embodiments, each of the one or more fluorescently labeled primers is associated with a different fluorophore.

Aspects of the present disclosure includes a method of determining the presence or absence of an aneuploidy, the method comprising: mixing a DNA sample of a subject and a plurality of spike-in molecules to create a mixture, each of the plurality of spike-in molecules associated with a chromosome of a set of chromosomes, wherein each of the plurality of spike-in molecules comprises: a target region having a first nucleotide sequence with sequence similarity to a target sequence region of the respective chromosome, a variation region having a second nucleotide sequence with sequence dissimilarity to a sequence region of the respective chromosome, and co-amplifying the mixture with one or more chromosome-specific primers to create a co-amplified mixture, wherein the one or more chromosome-specific primers are fluorescently labeled primers; receiving peak data from the co-amplified mixture, the peak data including, for each chromosome of the set of chromosomes, genomic peak intensities of the DNA sample and spike-in peak intensities of the spike-in molecules associated with the respective chromosome; for each chromosome, computing a ratio between the respective genomic peak intensity and the respective spike-in peak intensity; determining the presence or absence of the aneuploidy based on the computed ratios.

In some embodiments, the one or more chromosome-specific primers includes a set of chromosome-specific primers, each chromosome-specific primer in the set configured to capture a respective chromosome with a tail of a discrete length of a set of discrete lengths.

In some embodiments, computing, for each chromosome, the ratio between the respective genomic peak intensity and the respective spike-in peak intensity comprises: computing, for each discrete length of the set of discrete lengths, a ratio between the respective genomic peak intensity and the spike-in peak intensity; aggregating of the computed ratios across each discrete length of the set of discrete lengths.

In some embodiments, computing, for each chromosome, the ratio between the respective genomic peak and the respective spike-in peak intensity comprises: aggregating the genomic peak intensities across each discrete length of the set of discrete lengths; aggregating the spike-in peak intensities across each discrete length of the set of discrete lengths; computing a ratio between the aggregated genomic peak intensity and the aggregated spike-in peak intensity.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

Aspects of the present disclosure includes a method comprising: mixing a nucleic acid sample of a subject and a spike-in molecule associated with an allele to create a mixture, wherein the spike-in molecule includes a spike-in sequence, wherein the spike-in sequence comprises: a target region having a nucleotide sequence with sequence similarity to a target sequence region of the allele, a variation region having a nucleotide sequence with sequence dissimilarity to a sequence region of the allele, co-amplifying the mixture with one or more sets of allele-specific primers to generate a co-amplified mixture, each primer in a set of allele-specific primers configured to capture the respective allele and add a tail with a discrete length of a set of discrete lengths to an amplicon of the genomic sample and add a tail with the discrete length of the set of discrete lengths to an amplicon of the spike-in molecule, the amplicon of the genomic sample including the target sequence, the amplicon of the spike-in molecule including the spike-in sequence; labeling the co-amplified mixture with one or more fluorescently labeled primers; receiving peak data from the co-amplified mixture, the peak data including for each discrete length of the set of discrete lengths, genomic peak intensities of the genomic sample and spike-in peak intensities of the spike-in molecule; for each respective discrete length, computing a ratio between the respective genomic peak intensity and the spike-in peak intensity; and determining the presence or absence of the allele based on the computed ratios.

In some embodiments, computing, for each allele, the ratio between the respective genomic peak intensity and the respective spike-in peak intensity comprises: computing, for each discrete length of the set of discrete lengths, a ratio between the respective genomic peak intensity and the spike-in peak intensity; aggregating of the computed ratios across each discrete length of the set of discrete lengths.

In some embodiments, computing, for each allele, the ratio between the respective genomic peak intensity and the respective spike-in peak intensity comprises: aggregating the genomic peak intensities across each discrete length of the set of discrete lengths; aggregating the spike-in peak intensities across each discrete length of the set of discrete lengths; computing a ratio between the aggregated genomic peak intensity and the aggregated spike-in peak intensity.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

In some embodiments, each of the one or more fluorescently labeled primers is associated with a different fluorophore.

Aspects of the present disclosure include a method of determining the presence or absence of an aneuploidy, the method comprising: for each chromosome in a set of chromosomes: mixing a DNA sample of a subject and a spike-in molecule of a plurality of spike-in molecules to create a mixture, each of the plurality of spike-in molecules associated with the chromosome of the set of chromosomes, wherein each of the plurality of spike-in molecules comprises: a target region having a first nucleotide sequence with sequence similarity to a target sequence region of the respective chromosome, a variation region having a second nucleotide sequence with sequence dissimilarity to a sequence region of the respective chromosome, co-amplifying the mixture with one or more primers to generate a co-amplified mixture, each primer configured to capture a respective chromosome; for each length of a set of discrete lengths, adding a tail with the discrete length to a subset of amplicons in the co-amplified mixture; labeling the co-amplified mixture with one or more fluorescently labeled primers; receiving peak data from the co-amplified mixture, the peak data including genomic peak intensities of the portion of the DNA sample for each discrete length of the set of discrete lengths and the spike-in peak intensities of the spike-in molecule for each discrete length of the set of discrete lengths; for each respective discrete length, computing a discrete length-specific ratio between the respective genomic peak intensity and the spike-in peak intensity; aggregating the discrete length-specific ratios across each of the discrete lengths in the set of discrete lengths to generate a chromosome-specific ratio; and determining the presence or absence of aneuploidy based on the computed chromosome-specific ratios.

In some embodiments, determining the presence or absence of an aneuploidy based on the computed chromosome-specific ratios comprises: computing the ratio of a chromosome-specific ratio to each of the other chromosome-specific ratios; in response to determining a computed ratio is greater than a threshold ratio, determining the presence of aneuploidy; and in response to determining a computed ratio is less than a threshold ratio, determining the absence of aneuploidy.

In some embodiments, the variation region includes an insertion of base pairs with a length of: one base pair, two base pairs, three base pairs, four base pairs, or five base pairs.

In some embodiments, the variation region includes a deletion of base pairs with a length of: one base pair, two base pairs, three base pairs, four base pairs, or five base pairs.

In some embodiments, a location of a respective variation region of a spike-in molecule is in the center of a respective amplicon of the spike-in molecule.

Aspects of the present disclosure include a method of determining the abundance of endogenous targets, the method comprising: mixing a nucleic acid sample of a subject and a plurality of spike-in molecules to create a mixture, each of the plurality of spike-in molecules are associated with an endogenous target or targets, wherein each of the plurality of spike-in molecules further comprises: a target region having a first nucleotide sequence with sequence similarity to a target sequence region; a variation region having a nucleotide sequence with sequence dissimilarity to the target sequence; and co-amplifying the mixture with target specific primers to create a co-amplified mixture; labeling the co-amplified mixture by fluorescently labeled primers; receiving peak data from the co-amplified mixture, the peak data including, for each target of the set of targets, peak intensities of the nucleic acid sample and spike-in peak intensities of the spike-in molecules associated with each respective target; for each target, computing a ratio between the respective target peak intensity and the respective spike-in peak intensity; determining the abundance of the target based on computed ratios.

### DETAILED DESCRIPTION

Aspects of the present disclosure include methods of quantitative analysis of nucleic acid species suitable for applications such as, but not limited to, non-invasive prenatal testing (NIPT) and cancer detection, for a more accurate diagnosis of disorders and disease conditions.

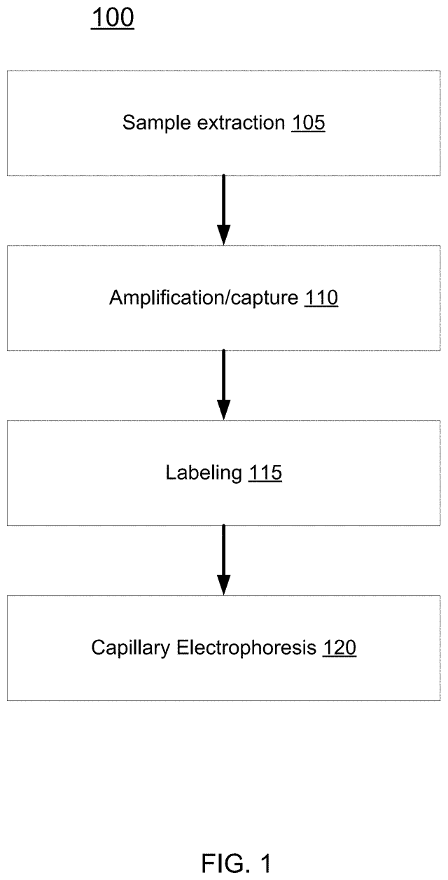

FIG. 1 is a flowchart of a method 100 of fragment analysis using spike-in molecules, according to one embodiment. Spike-in molecules are artificial molecules, designed based on the biological targets, such as the biological targets of chromosomes, alleles, etc. Quantitative data may be captured during fragment analysis through the use of spike-in molecules. For example, the use of spike-in molecules in known abundances can inform absolute abundances of a biological target in a genomic sample and/or relative abundances in a genomic sample. Fragment analysis is also described in U.S. Application Publication No. 2021/0292829, which is hereby incorporated reference in its entirety. The methods described herein may be used to more accurately detect aneuploidy and/or single gene disorders during noninvasive prenatal testing (NIPT). Examples of detectable gene and chromosome disorders include, but are not limited to, sickle-cell disease, cystic fibrosis, spinal muscular atrophy, beta-thalassemia, alpha-thalassemia, Patau syndrome, Down syndrome, Edwards syndrome, Turner syndrome, or the like.

Spike-in molecules have identical primer binding sites to a target sequence of a biological target, such as a chromosome or allele. Spike-in molecules include a spike-in sequence with a target region and a variation region. The target region includes a nucleotide sequence with sequence similarity (e.g., 100% sequence identity) to a target sequence of a biological target, such as a chromosome or allele. The variation region includes a nucleotide sequence with sequence dissimilarity to the target sequence of the biological target. The variation region differentiates the target sequences extracted from the biological target in a genomic sample from the spike-in sequences in the spike-in molecules such that the target sequences and the spike-in sequences are distinguishable during downstream processes. In some embodiments, the variation region is a deletion of one or more bases relative to the target sequence such that the lengths of the spike-in sequences and the target sequences vary. For example, the variation region of a spike-in molecule may include a four base deletion. In this example, where a target sequence includes 60 bases, the corresponding spike-in sequence includes 56 bases. Alternatively, or additionally, the variation is an insertion of one or more bases relative to the parget sequence. For example, the variation region of a spike in molecule may include a four base insertion. In this example, where a target sequence includes 60 bases, corresponding the spike-in sequence includes 64 bases.

The location of the variation region may vary. In some embodiments, the variation region is located within the center of the amplicon of the spike-in molecule, at an end of the amplicon, or the like. In addition, the spike-in molecule may include more than one variation region, such as two variation regions, three variation regions, etc., based on the disorder being detected. For example, in the detection of cystic fibrosis, two types of spike-in molecules may be used. The first spike-in molecule may be associated a wild type (WT) allele and include a single variation region. The second spike-in molecule may be associated with a F508del allele and may include two variation regions. For example, a first variation region may account for the 3-base deletion of the phenylalanine 508 (F508del) in the cystic fibrosis transmembrane conductance regulator and the second variation region distinguishes the second spike-in molecule from the first spike-in molecule.

In the method 100 shown in FIG. 1, a genomic sample is extracted 105. The sample may be a DNA sample. Alternatively, the sample may be an RNA sample or any other nucleotide model. Genomic samples are extracted through any appropriate sample extraction mechanism. A spike-in molecule associated with a biological target is mixed with the extracted sample. The mixture of the genomic sample and the spike-in molecule are captured and amplified 110. Amplification may be performed via any suitable mechanism, such as polymerase chain reaction (PCR), reverse-transcription PCR (PT-PCR), hybridization, ligation, or any other mechanism to measure molecules.

In some embodiments, this is an initial capture. During the initial capture, various primers may be used to reuse and/or resample amplicons, measure multiple amplicons simultaneously, or the like, which may help reduce noise. For example, primers may be used to tag amplicons with different fluorophores such that the same amplicon may be measured across different color channels. Data can then be aggregated for the same amplicon across the different channels to reduce noise. Similarly, primers may be used to add tails of different lengths to an amplicon such that the same amplicon may be measured multiple times across one or more color channels. For example, tails with a length of zero bases, six bases, twelve bases, eighteen bases, twenty-four bases, and the like, may be added to the amplicons of the target sequence and the spike-in sequence associated with the same biological target.

Moreover, primers may be used to measure multiple separate amplicons simultaneously. In one embodiment, multiple separate amplicons may be measured simultaneously by labeling separate amplicons with different fluorophores. For example, for a first chromosome, the target sequences and corresponding spike-in sequences may be labeled with a fluorophore that emits blue light. For a second chromosome, the target sequences and corresponding spike-in sequences may be labeled with a fluorophore that emits red light. Alternatively, or additionally, tails of various lengths may be added to the amplicons corresponding to each chromosome, each of which has been tagged with a different fluorophore. Thus, the amplicons of various sizes may be aggregated across each size but within a color channel. This enables multiple separate amplicons to be measured simultaneously while resampling, which may reduce noise.

When there is an initial capture, the amplified mixture is labeled 115 with fluorescently labeled primers. The amplified mixture 115 may be labeled via an additional amplification step, such as with PCR. In other embodiments, the mixture of the extracted sample and one or more spike-in molecules is directly amplified with fluorescently labeled primers such that there is a single amplification and labeling step. Alternatively, there may be greater or fewer amplification steps based on the application.

Capillary electrophoresis 120 is performed on the amplified and labeled mixture. Any suitable capillary electrophoresis protocol may be used. Data, such as peak data, is received from the capillary electrophoresis. Data may be aggregated in any suitable manner across size and color channels. The data of both the genomic sample and the spike-in molecules may be used to determine absolute and relative abundances of the biological target in the genomic sample. Absolute abundances may be estimated by comparing the data of the sample peaks to spike-in peaks. Relative abundances of alleles may be estimated if the alleles differ in length. The ratio of the spike-in peaks and the sample peaks may be used to estimate dosage, discussed in detail below.

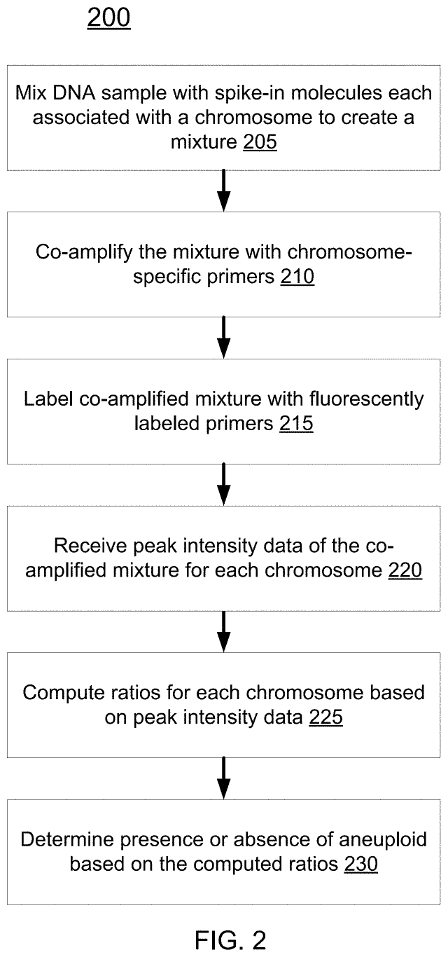

FIG. 2 is a flowchart of a method 200 of aneuploidy detection, according to one embodiment. In the method 200 shown, a DNA sample of a subject and spike-in molecules are mixed 205 to create a mixture. Each spike-in molecule is associated with a chromosome, such as Chromosome 13, Chromosome 18, Chromosome 21, Chromosome X, Chromosome Y, or the like. The mixture is co-amplified 210 with chromosome-specific primers to create a co-amplified mixture. In some embodiments, the chromosome-specific primer is a forward primer. In these embodiments, a universal reverse primer may be used. In alternative embodiments, the chromosome-specific primer is the reverse primer and a forward primer is a universal primer; both the forward primer and reverse primer are chromosome-specific primers, or the like. The co-amplified mixture is labeled 215 with fluorescently labeled primers. The labeled co-amplified mixture undergoes capillary electrophoresis. Peak data is received 220 from the capillary electrophoresis. Any suitable capillary electrophoresis protocol may be used, such as using a fragment analysis mode. In some embodiments, the peak data includes, for each chromosome, genomic peaks intensities of the target sequences and spike-in peak intensities of the spike-in sequences.

Data may then be aggregated based on the primers used during amplification to compute ratios 220 between the respective genomic peak intensity of the target sequence and the respective spike-in peak intensity of the spike-in sequence for each chromosome. The presence or absence of aneuploidy is determined 230 based on the computed ratios. In some embodiments, aneuploid is predicted 230 by computing the ratio of a chromosome-specific ratio to each of the other chromosome-specific ratios. For example, the ratio of the target sequence to the spike in sequence is computed for each chromosome, such Chromosome 13, Chromosome 18, Chromosome 21, Chromosome X, and Chromosome Y. Then, ratios between a particular chromosome-specific ratio and each of the other chromosome-specific ratios are computed. For example, in determining the presence or absence of an aneuploid, the Chromosome 13: Chromosome 18 ratio, Chromosome 13: Chromosome 21 ratio, Chromosome 13: Chromosome X ratio, and Chromosome 13: Chromosome Y ratio are computed. An aneuploid may be predicted based on a comparison of these ratios. For example, an aneuploid may be predicted when a computed ratio is greater than a threshold ratio, such as greater than one half the fetal fraction. Similarly, a euploid may be predicted when a computed ratio is less than a threshold ratio, such as around unity.

As an example, if a fetus is contributing more than two copies of a chromosome to a maternal cell-free DNA sample, the fragments from that chromosome will be in excess compared to fragments from other chromosomes. In an embodiment, this is detected by measuring aneuploidy of a number of chromosomes against a chromosome that is known to not be aneuploid. For example, Chromosome 13, Chromosome 18, and Chromosome 21 may be compared to Chromosome 1. A direct comparison of chromosomes may not be possible because each region may amplify differently, as represented by different multiplication factors, A and B, in Equation 1. Thus, spike-in molecules may act as a normalization factor for each region being amplified. In an embodiment, if equal amounts of spike-in molecules for each chromosome are used, in a euploid, the ratios of the spike-in molecules to target molecules are equal across different chromosomes. For example, in a euploid, the ratio between the output generated from Chromosome 21 target in the DNA sample and that generated from Chromosome 21 spike-in molecules is equal to the ratio the between the output generated from Chromosome 1 target in the DNA sample and the Chromosome 1 spike-in molecules, in accordance with Equation 1.

$$\frac{A * Chromosome 21 target}{A * Chromosome 21 spike - in} = \frac{B * Chromosome 1 target}{B * Chromosome 1 spike - in} \qquad ( 1 )$$

Each of the four numerators and denominators above are measurable. Further, because equal amounts of both the Chromosome 21 spike-in molecules and Chromosome 1 spike-in molecules are used, Equation 1 becomes Equation 2 when it is euploid.

Chromosome 21==Chromosome 1 (2)

In some embodiments, by using the same color or length, as discussed above, the signals from many fragments on the same chromosome may be aggregated into the same intensity peak. In these embodiments A*Chromosome 21 target becomes one intensity peak, A*Chromosome 21 spike-in becomes one intensity peak, B*Chromosome 1 target becomes one intensity peak, and B*Chromosome 1 spike-in becomes one intensity peak. Alternatively, the signals may be measured across a plurality of peaks for a given chromosome and averaged.

Alternatively, or additionally, aneuploidy may be detected without the use of a chromosome known to not be aneuploid. In these embodiments, the ratio of the target molecules to spike-in molecules of a first chromosome is compared to the ratio of the target molecules to the spike-in molecules of a second chromosome. If the ratio corresponding to the first chromosome is significantly greater than a ratio corresponding to the second chromosome, the fetus likely has aneuploid at the first chromosome. For example, where there is a presence of Down Syndrome, the left-hand side of Equation 3 will be significantly higher than the right-hand side (given a fetal fraction). If the fetal fraction is 10%, it is expected that the left-hand side ratio is 5% higher than right-hand side ratio. This is because 90% of the maternal DNA with two copies of Chromosome 21 plus 10% of the fetal DNA with three copies of Chromosome 21 leads to an overall 5% increase in the number of endogenous target molecules of Chromosome 21 origin. Significance of this excess may be calculated as a z-score, a likelihood ratio, or any suitable metric, to determine the likelihood the fetus has aneuploid.

$$\frac{A * Chromosome 21 target}{A * Chromosome 21 spike - in} > \frac{B * Chromosome 18 target}{B * Chromosome 18 spike - in} \qquad ( 3 )$$

As another example, where there is a presence of Edwards Syndrome, the right-hand side of Equation 4 will be significantly higher than the left-hand side.

$$\frac{A * Chromosome 21 target}{A * Chromosome 21 spike - in} < \frac{B * Chromosome 18 target}{B * Chromosome 18 spike - in} \qquad ( 4 )$$

In some embodiments, a set of chromosome-specific primers may be used for each chromosome to reuse and/or resample the same molecules and/or reduce noise. In some embodiments, each primer in the set is configured to capture a respective chromosome with a tail of a discrete length of a set of discrete lengths. Tails may be introduced as a reverse label tail. Tails may be any suitable length, such as between 0 base and 100 bases. For example, a primer that adds a 6-base tail will generate an amplicon with 6 additional bases. Similarly, a primer that adds an 8-base tail will generate an amplicon with 8 additional bases. Thus, peak intensity data for a single chromosome and/or allele may be aggregated across each of the sizes.

For example, a set of primers associated with Chromosome 13 may include four primers that each add a tail of a discrete length to the corresponding amplicons. Tail lengths may include tails with zero bases, 6 bases, 12 bases, and 18 bases. There, capillary electrophoresis will generate peak data for Chromosome 13 for the target sequences and spike-in sequences at each of the four lengths. The peak data for Chromosome 13 may then be aggregated across each of the sizes. Any suitable data metric may be used, including, but not limited to, the mean of each peak, the median of each peak, the maximum of each peak, the minimum of each peak, or the like. Alternatively, or additionally, each primer in the set may be associated with a different color channel such that each primer captures a respective chromosome and adds a color-specific tag to a set of target sequences and spike-in sequences associated with the chromosome. Thus, peak data for a single chromosome may be aggregated across each of the color channels with any suitable technique and/or metric.

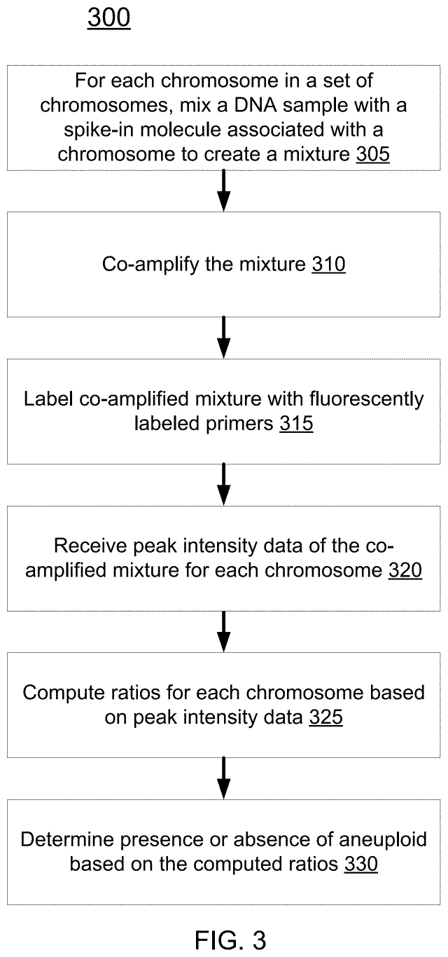

FIG. 3 is a flowchart of an alternative method 300 of aneuploidy detection, according to one embodiment. In this method 300, individual iterations of capillary electrophoresis may be run for each chromosome in a set of chromosomes. As shown, for each chromosome, a DNA sample of a subject and a spike-in molecule associated with a chromosome are mixed 305 to create a mixture. The mixture is co-amplified 310 with one or more primers. In some embodiments, the one or more primers are fluorescently labeled primers. In alternative embodiments, the mixture undergoes an initial capture step in which tails and/or tags are added to the amplicons during amplification 310 that enable additional techniques to be used downstream. As discussed above, in some embodiments, each of the one or more primers adds a tail to the amplicon of the target sequence and the spike-in sequence. The tail may add bases to the amplicons. Alternatively, or additionally, the tag may add a color-specific label to each of a subset of target sequences and spike-in sequences. The amplified mixture is labeled 315 with fluorescently labeled primers. Capillary electrophoresis is performed on the labeled co-amplified mixture. Peak intensity data of the co-amplified mixture is received 320. Ratios between the genomic peak intensities and the spike-in peak intensities are computed 325 for each chromosome. The presence or absence of aneuploid is determined 330 based on the chromosome-specific ratios using the methods described above.

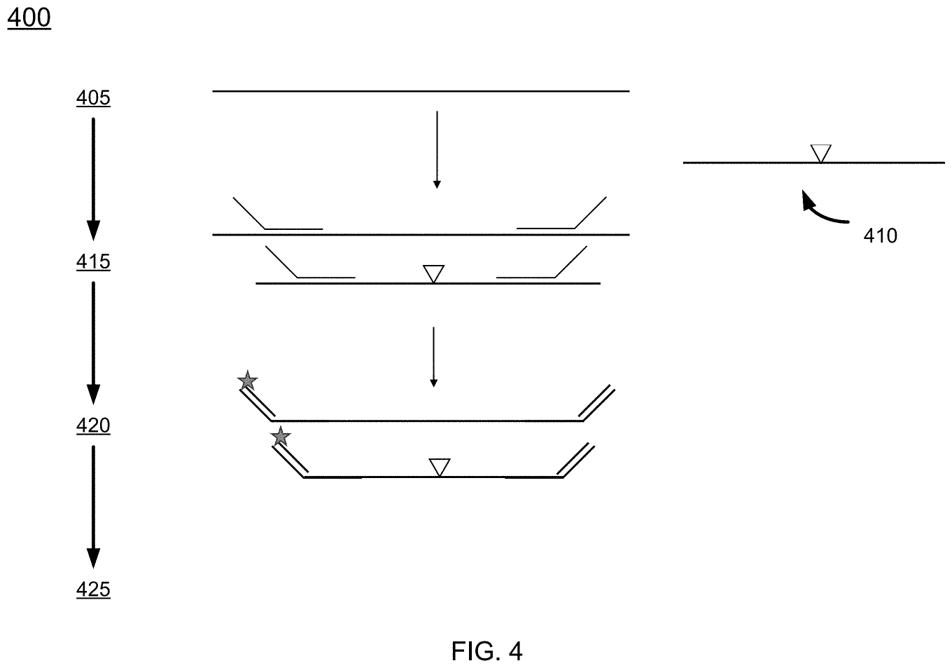

FIG. 4 is a is a block diagram 400 illustrating an example of aneuploidy detection, according to one embodiment. In the block diagram shown, purified cfDNA 405 is mixed with a predetermined number of spike-in molecules 410, such as 5000 copies with a four base pair deletion relative to the target sequence with one per locus. Multiplex PCR 415 is performed with 100 to 250+ per chromosome, adding chromosome-specific tails to the molecules. Fluorescent labels 420 are added to each chromosome, and the labeled chromosomes undergo capillary electrophoresis 425.

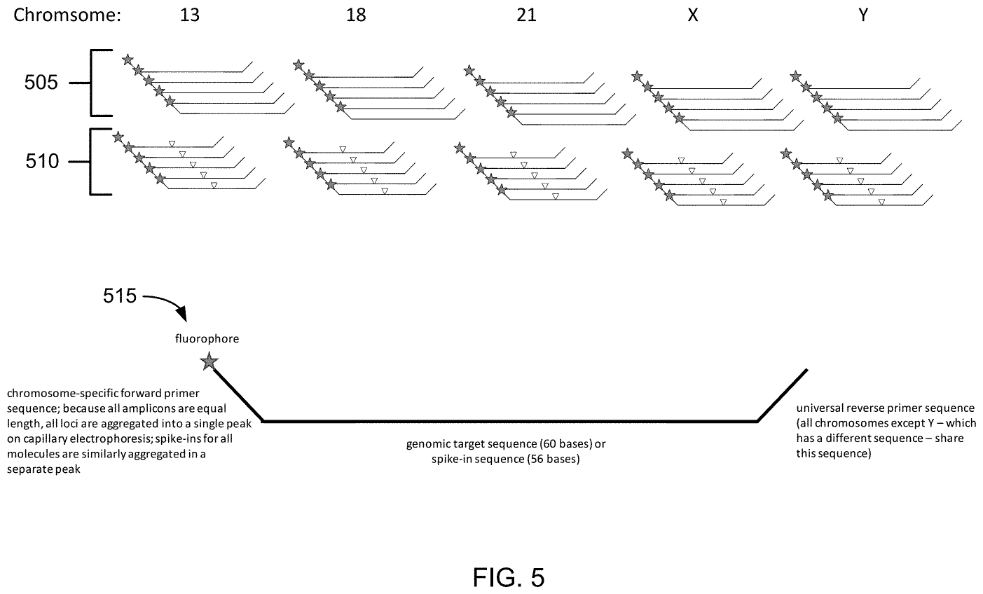

FIG. 5 is a block diagram 500 illustrating amplified target sequence molecules 505 and amplified spike-in sequence molecules 510, according to one embodiment. Five chromosomes are shown in the block diagram 500, namely Chromosome 13, Chromosome 18, Chromosome 21, Chromosome X, and Chromosome Y. In alternative embodiments, greater, fewer, and/or different chromosomes may be used. A magnified sequence 515 is also shown, which may represent either a target sequence or a spike-in sequence. In the magnified sequence 515, the genomic target sequence has a length of 60 bases and the corresponding spike-in sequence as 56 bases (4 base pair deletion). Further, the magnified sequence 515 includes a chromosome-specific forward primer and a universal reverse primer sequence.

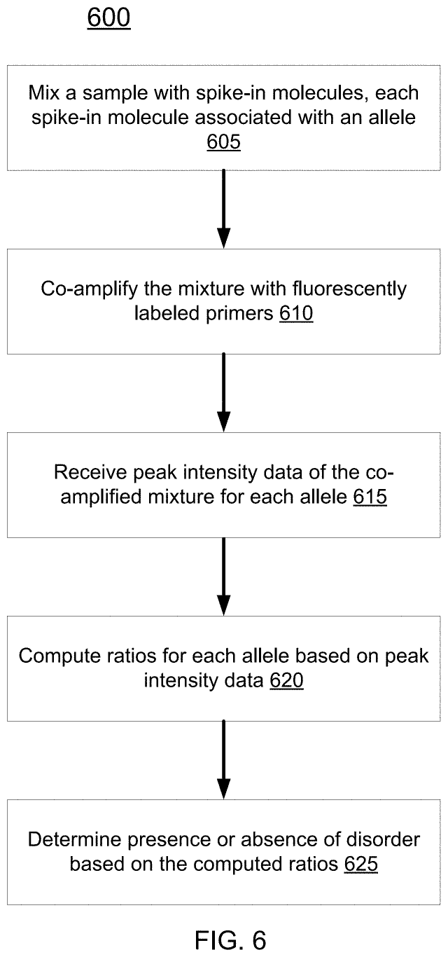

FIG. 6 is a flowchart of a method 600 of single gene disorder detection, according to one embodiment. In the method 600 shown, a genomic sample is mixed 605 with spike-in molecules. Each spike-in molecule is associated with an allele and includes a spike-in sequence. The spike-in sequence includes a target region with sequence similarity to a target sequence of a corresponding allele and a variation region with sequence dissimilarity to the target sequence of the corresponding allele. The mixture is co-amplified 610 with fluorescently labeled primers. The mixture undergoes capillary electrophoresis. Peak intensity data of the co-amplified mixture is received 615. Ratios are computed 620 for each allele based on the peak intensity data of the target sequences and the spike-in sequences. The presence or absence of the single-gene disorder is determined 625 based on the computed ratios.

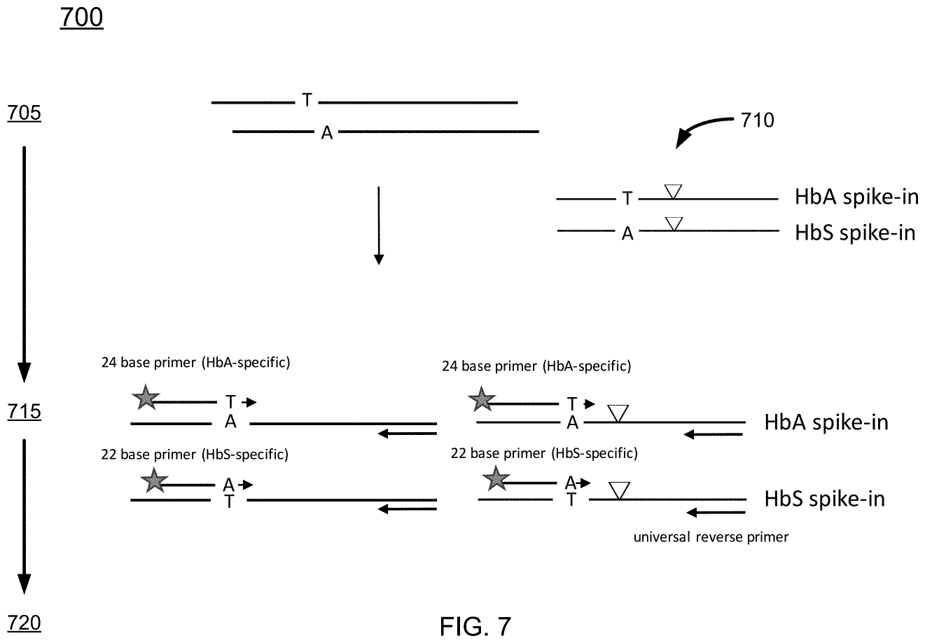

FIG. 7 is a block diagram 700 illustrating an example of sickle cell detection, according to one embodiment. In one embodiment, cfDNA is extracted from a sample 705. Spike-in molecules 710 are mixed with the sample 705. In the embodiment shown, a spike-in molecule associated with HbA and a spike-in molecule with HbS are added. In an embodiment, one spike-in per allele is added. Each spike-in may include an insertion or a deletion, such as a four base-pair deletion. The number of copies of each spike-in may vary. For example, there may be any suitable number of copies, including, but not limited to 500 copies, 1000 copies, 2000 copies, 5000 copies, 10,000 copies, or the like.

Fluorescently labeled allele-specific primers are used to capture, amplify, and label 715 the amplicons. In some embodiments, each allele-specific primer captures each allele, but generates different length amplicons such that alleles are distinguishable during capillary electrophoresis and data aggregation. For example, HbA-specific primers may generate HbA amplicons are of a different length than the HbS amplicons generated by HbS-specific amplicons. The difference in length may be any suitable number of bases, such as 1 base, 2 bases, 3, bases, 4 bases, 5 bases, 10 bases, 20 bases, etc. For example, the HbA-specific primer may generate HbA amplicons with target sequences that are 74 bases and spike-in sequences that are 70 sequences, and the HbS-specific primer may generate HbS amplicons with target sequences that are 72 bases and spike-in sequences that are 68 bases. Capillary electrophoresis is performed 720 on the amplified and labeled mixture. Each molecule will appear as a peak in capillary electrophoresis. Molecule counts may be estimated by computing the ratios of intensities for genomic peaks of the target sequences and the spike-in peaks of the spike-in sequences for each allele. The relative allele fractions may be computed by comparing the ratios of genomic peak intensities of the target sequences to spike-in peak intensities of the spike-in sequences across alleles.

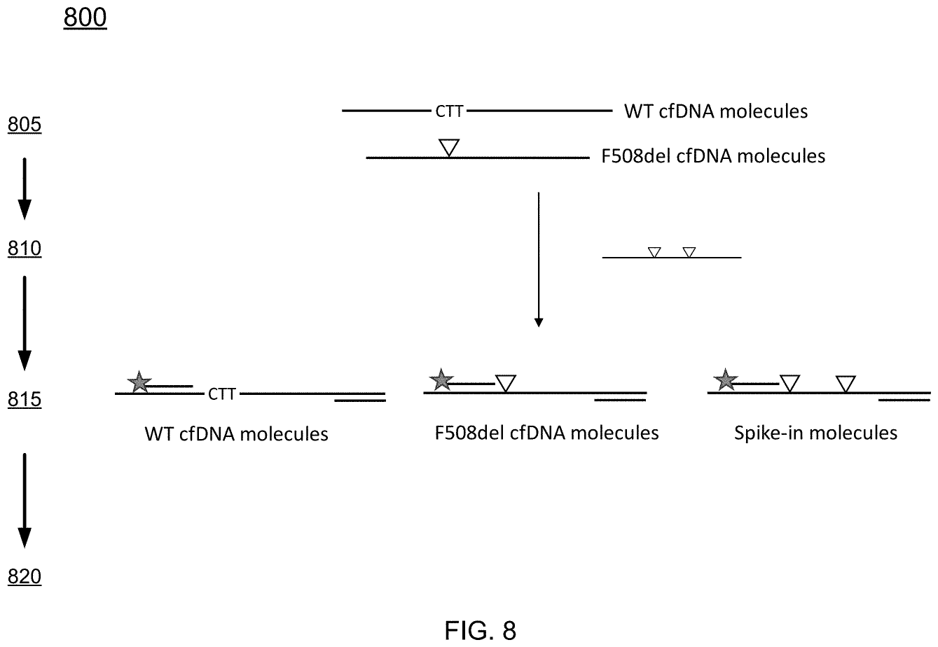

FIG. 8 is a block diagram 800 illustrating an example of cystic fibrosis detection, according to one embodiment. In one embodiment, cfDNA is extracted from a sample 805. A single spike-in molecule is mixed 810 with the sample. The spike-in includes a first variation region with a four base deletion and a second variation region with an additional deletion to estimate molecule counts. For example, the second variation region may account for the 3-base deletion of phenylalanine 508 (F508del) in exon 11. Fluorescently labeled primers amplify across the deletion site, which generates different length amplicons for WT molecules, F508del molecules, and the spike-in molecules. For example, the target sequence of the WT molecule may include 76 bases, the target sequence of the F508del molecule may include 73 bases, and the spike-in sequence of the spike-in molecule may include 69 bases. Capillary electrophoresis is performed 820 on the amplified and labeled mixture. Each molecule will appear as a peak in capillary electrophoresis. The relative allele fractions can be computed by comparing the intensities across peaks for each allele.

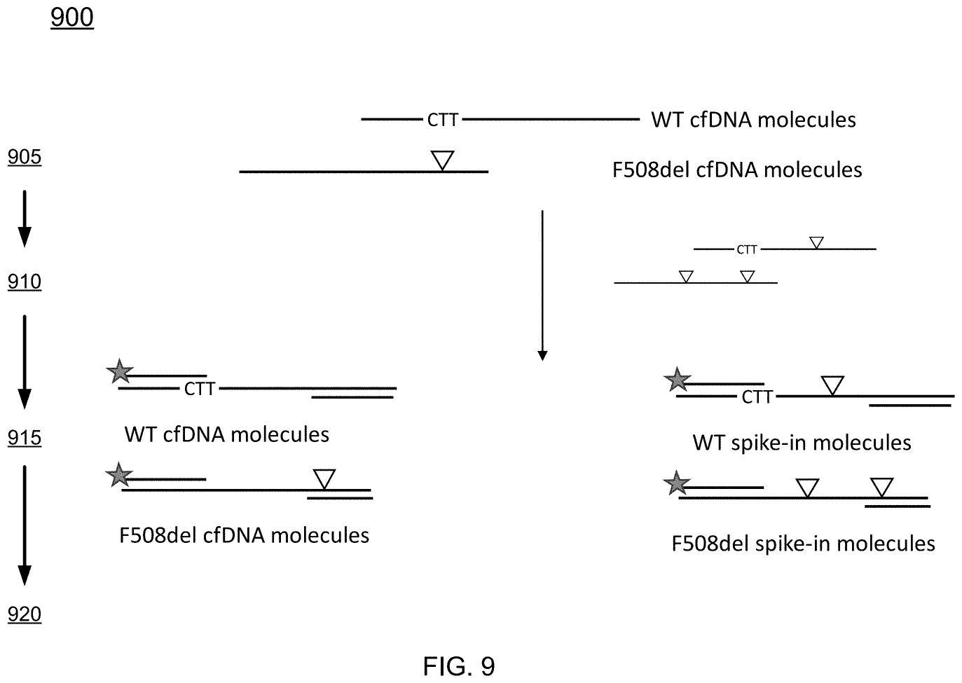

FIG. 9 is a is a block diagram 900 illustrating an additional example of cystic fibrosis detection, according to one embodiment. In one embodiment, cfDNA is extracted 905 from a sample. A spike-in molecule associated with a WT allele (e.g., a spike-in molecule with a single variation region) and a spike-in molecule associated with a F508del molecule (e.g., a spike in molecule with multiple variation regions) is mixed 910 with the sample. In some embodiments, one spike-in per allele is added. The number of copies of each spike-in may vary. For example, there may be any suitable number of copies, including, but not limited to 500 copies, 1000 copies, 2000 copies, 5000 copies, 10,000 copies, or the like. Fluorescently labeled primers specifically capture 915 each allele but generate different length amplicons. For example, the WT-primer may generate WT amplicons with target sequences with 89 bases and WT spike-in amplicons with spike-in sequences with 85 sequences. Similarly, the F508del-primer may generate F508del amplicons with target sequences with 95 bases and F508del spike-in amplicons with target sequences with 91 sequences. In some embodiments, amplification 915 is performed with allele-specific PCR by placing a 3′ primer end in the deletion (e.g., WT-specific) or across the deletion with two anchoring bases (e.g., F508del-specific) labeled primers. The capillary electrophoresis is performed 920 on the amplified and labeled mixture. Each molecule will appear as a peak in capillary electrophoresis. Molecule counts can be estimated by computing the ratios of intensities for genomic and spike-in peaks for each allele. Alternatively, or additionally, relative allele fractions can be computed by comparing the ratios of genomic peak intensities to spike-in peak intensities across alleles.

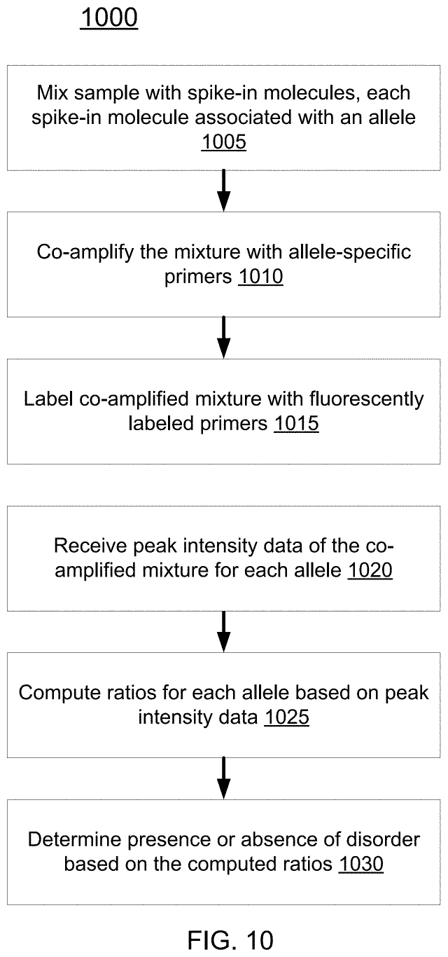

FIG. 10 is a flowchart of an alternative method 1000 of single gene disorder detection, according to one embodiment. In the method 1000 shown, a genomic sample is mixed 1005 with spike-in molecules. Each spike-in molecule is associated with an allele and includes a spike-in sequence. The spike-in sequence includes a target region with sequence similarity to a target sequence of a corresponding allele and a variation region with sequence dissimilarity to the target sequence of the corresponding allele. The mixture is co-amplified 1010 with allele-specific primers. As discussed with reference to FIG. 1, allele-specific primers may be used to reuse, resample, and/or measure multiple separate alleles simultaneously. For example, allele-specific primers may be used to tag different alleles with different fluorophores. Alternatively, or additionally, allele-specific primers may be used to add tails of different lengths to an amplicon such that the same amplicon may be measured multiple times across one or more color channels. The co-amplified mixture is labeled 1015 with fluorescently labeled primers. The mixture undergoes capillary electrophoresis. Peak intensity data of the co-amplified mixture is received 1020. Ratios are computed 1025 for each allele based on the peak intensity data of the target sequences and the spike-in sequences. The presence or absence of the single-gene disorder is determined 1030 based on the computed ratios.

Other valid conditions can include different sample types. In some embodiments the sample can be cell-free DNA. In some embodiments the sample is gDNA. In some embodiments the sample can be RNA (with modifications to protocol). In a preferred embodiment the same is cell-free DNA. In another preferred embodiment the sample is gDNA.

The sample volume can be 1-45 The spike-ins can include any addition, or deletion, of base pairs. In some embodiments the size of the spike-in is ±2 bps-±20 bps compared to the amplicon length. In some embodiments it is ±3 bps compared to the amplicon length. In some embodiments it is ±4 bps compared to the amplicon length. In some embodiments it is ±5 bps compared to the amplicon length. In some embodiments it can ±6 bps compared to the amplicon length. In some embodiments it is ±7 bps compared to the amplicon length. In some embodiments it is ±8 bps compared to the amplicon length. In some embodiments it is ±9 bps compared to the amplicon length. In some embodiments it is ±10 bps compared to the amplicon length. In some embodiments it is ±11 bps compared to the amplicon length. In some embodiments it is ±12 bp compared to the amplicon length. In some embodiments it is ±13 bps compared to the amplicon length. In some embodiments it is ±14 bps compared to the amplicon length. In some embodiments it is ±15 bps compared to the amplicon length.

There can be different type of spike-ins, including even more than one per target sequence (for example, chromosome). In some embodiments the number of different types of spike-ins is 1-5.

The number of copies of each spike-in/locus is a discrete number to allow for quantification. It can be any number. In some embodiments it is 500-200,000. In some embodiments it is 500-100000. In some embodiments it is 500-50,000. In some embodiments it is 250-25,000. In some embodiments it is 100-20,000. In some embodiments it is 500-10,000. In some embodiments it is 1,000-5,000.

The target amplicon can be any size. In some embodiments the target amplicon is used to measure chromosome aneuploidy. In some embodiments the target amplicon is used to measure copy number variation (CMV) on all or part of a chromosome. The amplicon can be any size. In some embodiments the amplicon is 30 bps-500 bps. In some embodiments the amplicon is 20 bps-450 bps. In some embodiments the target amplicon is 20 bs-400bps. In some embodiments the target amplicon is 30 bps-200 bps. In some embodiments the target amplicon is 50 bps-100 bps.

The amplicon count can be any number.

The PCR method steps can be varied by one skilled in the art, including varying primer concentrations, cycle count, annealing time, annealing temperature, extension time, dilution factor, labeling primer concentration, sample volume, and whether a size standard is present or absent.

To clarify the signal received from the capillary electrophoresis, the protocol may be altered. In some embodiments the noise is reduced as compared to an alternate protocol. In some embodiments the peaks are more resolved as compared to an alternate protocol.

In some embodiments the voltage for the injection may be modified. In some embodiments it may be 0.5-15 kV. In some embodiments it may be 0.5-10 kV. In some embodiments it may be 10-15 kV. In some embodiments it may be 0.1-5 kV. In some embodiments it may be 15 kilovolts (kV). In some embodiments it may be 7.5 kV. In some embodiments it may be 5 kV. In some embodiments it may be 4 kV. In some embodiments it may be 3 kV. In some embodiments it may be 2 kV. In some embodiments it may be 1 kV. In some embodiments it may be 0.5 kV.

In some embodiments the voltage for the run may be modified. In some embodiments it may be 0.5-15 kV. In some embodiments it may be 0.5-10 kV. In some embodiments it may be 10-15 kV. In some embodiments it may be 0.1-5 kV. In some embodiments the voltage may be 15 kilovolts (kV). In some embodiments it may be 7.5 kV. In some embodiments it may be 5 kV. In some embodiments it may be 4 kV. In some embodiments it may be 3 kV. In some embodiments it may be 2 kV. In some embodiments it may be 1 kV. In some embodiments it may be 0.5 kV.

In some embodiments the injection time may be modified. In some embodiments the exposure time is 50-1000 milliseconds (ms). In some embodiments the exposure time is 50-400 ms. In some embodiments the exposure time is 50-300 ms. In some embodiments the exposure time is 100-450 ms. In some embodiments the exposure time is 150-450 ms. In some embodiments the exposure time is 50 ms. In some embodiments the exposure time is 100 ms. In some embodiments the exposure time is 200 ms.

In some embodiments the injection time may be modified. In some embodiments the injection time is 1-24 seconds (s). In some embodiments the injection time is 2-10 s. In some embodiments the injection time is 2-8 s. In some embodiments the injection time is 3-6 s. In some embodiments the injection time is 3 s. In some embodiments the injection time is 4 s. In some embodiments the injection time is 5 s. In some embodiments the injection time is 6 s.

The sample can be reinjected between 1-co times, with noise decreasing as the number of reinjections increases. In some embodiments the sample is reinjected 1-100 times. In some embodiments the sample is reinjected 1-75 times. In some embodiments the sample is reinjected 1-50 times. In some embodiments the sample is reinjected 1-25 times. In some embodiments the sample is reinjected 1-15 times. In some embodiments the sample is reinjected 1-12 times. In some embodiments the sample is reinjected 1-11 times. In some embodiments the sample is reinjected 1-10 times. In some embodiments the sample is reinjected 1-9 times. In some embodiments the sample is reinjected 1-8 times. In some embodiments the sample is reinjected 1-7 times. In some embodiments the sample is reinjected 1-6 times. In some embodiments the sample is reinjected 1-5 times. In some embodiments the sample is reinjected 1-4 times. In some embodiments the sample is reinjected 1-3 times. In some embodiments the sample is reinjected 1-2 times. In some embodiments the sample is reinjected 1 time.

Targets may include genes, chromosomes, and fragments thereof; they may also include synthetic nucleic acid molecules for tracing or other purposes; they may also include RNA species and/or fragments thereof.

The methods herein can also be used to detect, quantify, and/or otherwise characterize molecules of a particular locus. It can be used to characterize microdeletions, microinsertions, copy number variations, and/or chromosomal abnormalities both for prenatal diagnostics and for liquid biopsies (and/or for any suitable conditions). Embodiments include quantification of copy number variants (CNVs) for applications in microdeletion detection in the prenatal setting and/or in a non-prenatal setting; quantification and/or detection of CNVs or SNVs in connection with cancer detection, monitoring, diagnosis, or quantification; detection, characterization, or quantification of breakpoints; quantification of nucleic acid fusions in cancer and other related diseases; gene expression quantification for cancer detection, monitoring, diagnosis, or quantification; gene expression quantification for non-cancer related purposes including infection monitoring, immune system monitoring, or detection, diagnosis or monitoring of any other condition. Other embodiments also include quantification of CNVs or SNVs in connection with infectious diseases, such as, but not limited to: influenza (e.g., Influenza A, Influenza B), Covid (e.g., SARS-CoV-2) detection, and the like. In some embodiments, the infectious disease is: coronavirus, influenza virus, rhinovirus, respiratory syncytial virus, metapneumovirus, adenovirus, or boca virus. In some embodiments, the influenza virus is: parainfluenza virus 1, parainfluenza virus 2, influenza A virus, or influenza B virus. In some embodiments, the coronavirus is: coronavirus 0C43, coronavirus 229E, coronavirus NL63, coronavirus HKU1, middle east respiratory syndrome beta coronavirus (MERS-CoV), severe acute respiratory syndrome beta coronavirus (SARS-CoV), or SARS-CoV-2.

The methods herein may provide absolute quantification. One embodiment of this is using a known number of spike-in molecules to compute the total number of target molecules. This method may provide relative quantification in cases where knowledge of relative abundance is desirable or absolute spike-in abundance is unknown. One embodiment is using two or more spike-ins for two or more targets and including these spike-ins at equal abundance. Ratios of target molecule to spike-in molecule measurements may be used to compare the relative abundance of each of these targets. Targets may represent one or more regions of interest. For instance, several targets within one gene (e.g. EGFR) might be used to compare its copy number to a reference target or targets (eg. an entire chromosome or chromosomes).

### EXAMPLES

### Example 1: Measurements of DNA

Aneuploidy measurement is performed on a cfDNA sample. cfDNA is extracted from plasma and purified.

The 36 μl of cfDNA is combined with spike-ins molecules (in this example 5000 and 10000 copies of a −6 base and +8 base spike-in, respectively) that control for amplification and primers that amplify hundreds (approximately 900 total) of 60 bp DNA loci across chromosomes of interest (in this example 13, 18, 21, X, and Y).

25 cycles of an initial amplification reaction are performed with 4:59 min annealing time and 60° C. annealing temperatures using Q5 polymerase.

The initial amplification is diluted for secondary amplification (1:50).

The diluted initial amplification is combined in 5 different reactions (one for each chromosome) with fluorescently labeled primers (5′FAM).

30 cycles of amplification are performed using 60° C. annealing temperature and 30 s annealing time using Q5 polymerase.

2 μl of the secondary PCR reaction is combined with size standard and formamide. The mixture is heated for 5 minutes at 95° C. and then rapidly cooled to 4° C.

The plate is injected on a 36 cm capillary array using a 3730×1 12 times using injection time 4 s, injection voltage 3 kV, run voltage 5 kV, and exposure times of 200 ms.

### Example 2: Noise Reduction

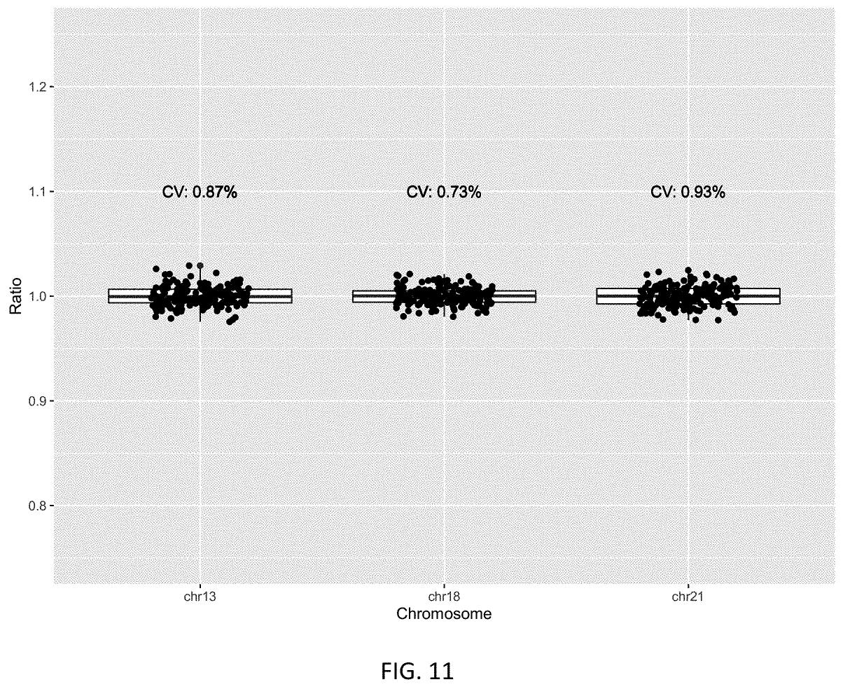

In order to achieve the needed results, noise needs to be reduced. FIG. 11 shows the total noise in the sample (both measurement and sample noise). A ratio of 1 is expected. The sample is extracted cfDNA from a euploid, pregnant subject. Assay was performed on each sample and ratios are calculated from averaging 12 injections and 2 spike-ins on capillary electrophoresis (CE).

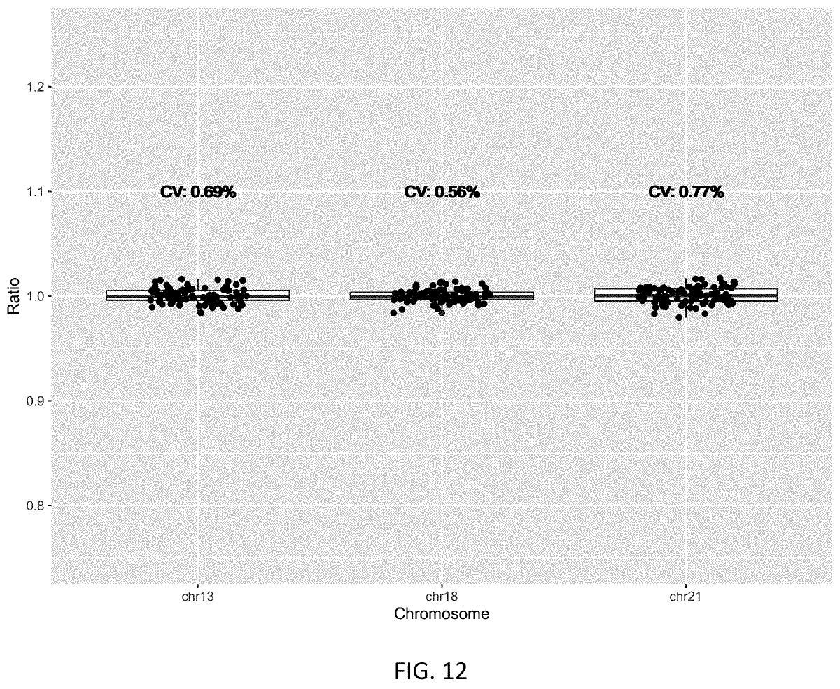

There are two sources of noise capture, which is noise that comes from the PCR required to prepare the sample for measurement, and measurement noise, which is noise inherent in measuring the sample. NGS removes the measurement noise but leaves the capture noise. FIG. 12 is the same sample as FIG. 11, but the assay was performed on each sample, followed by NGS sequencing to remove the measurement noise. Ratios were computed by summing reads to mimic capillary electrophoresis measurement.

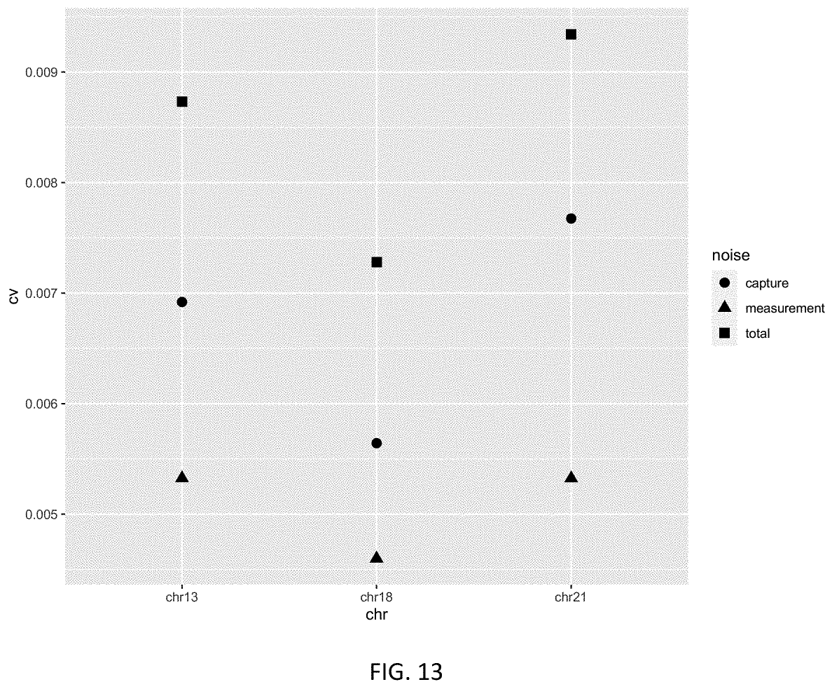

FIG. 13 shows the total noise in a sample, including the noise contributions from capture and measurement. Measurement noise was imputed by subtracting capture noise from NGS measurements from total noise in quadrature.

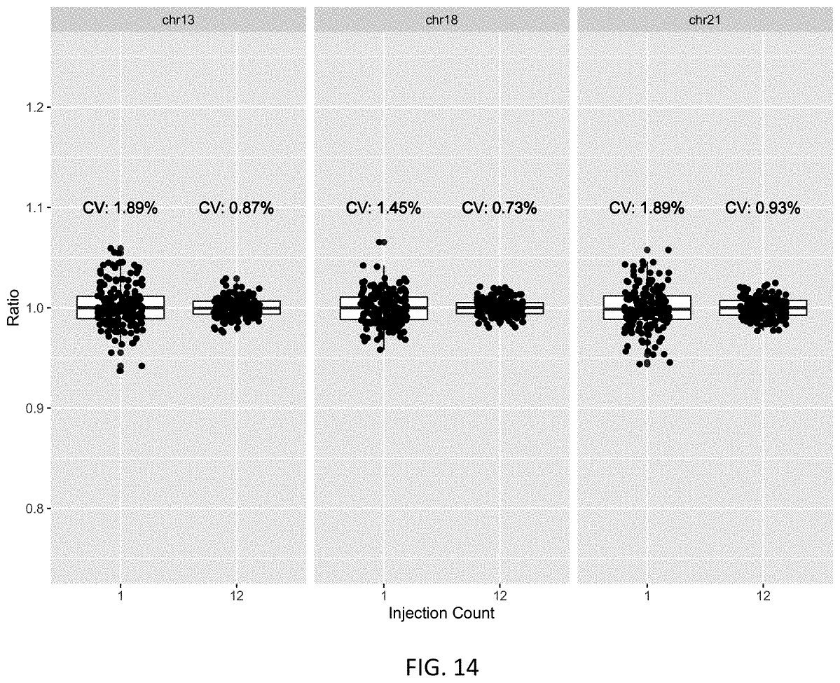

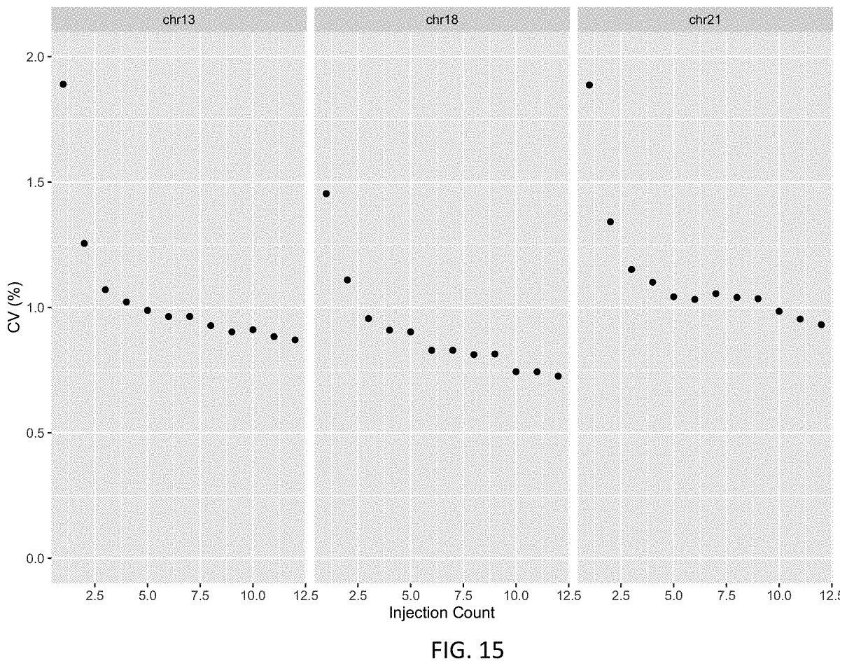

In order to reduce the noise from the instrument (measurement noise), the same sample was reinjected multiple times. There is a decrease in the variance as the number of reinjections increases. This trend continues towards the results of an NGS sample which has no instrument noise. FIGS. 14 and 15 show the decrease in noise as the injection number increases. At twelve reinjections the noise is about half as compared to one reinjection.

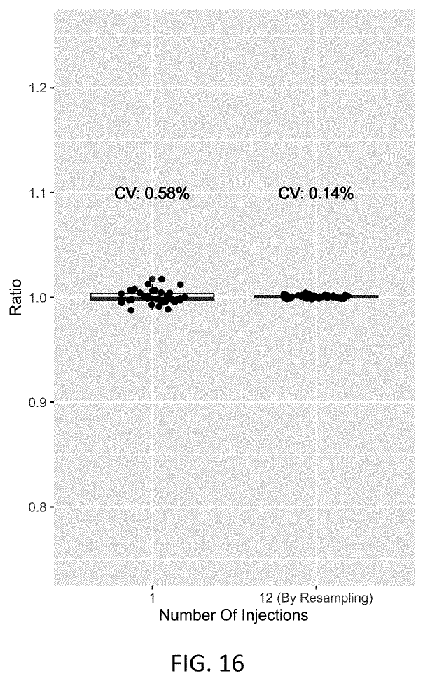

FIG. 16 shows how low the noise measurement can get when the methods for reducing noise are combined.

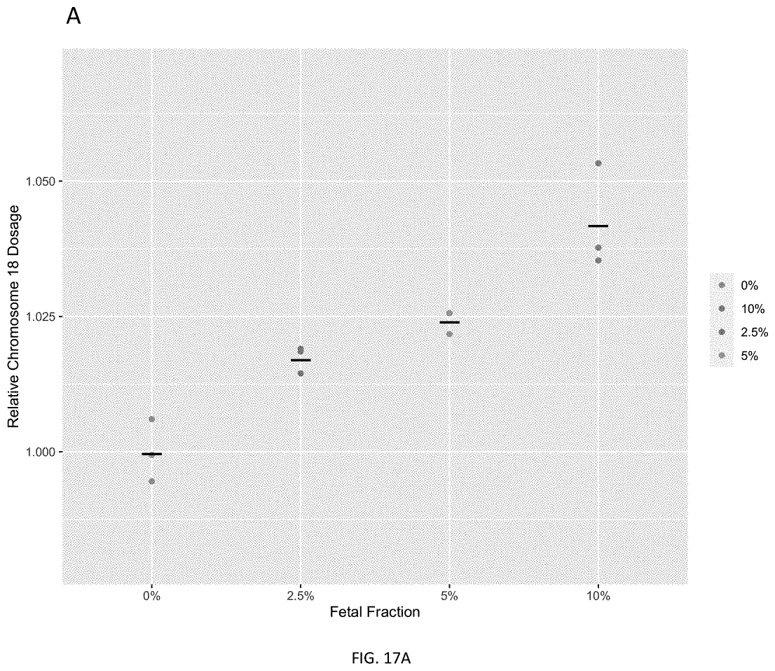

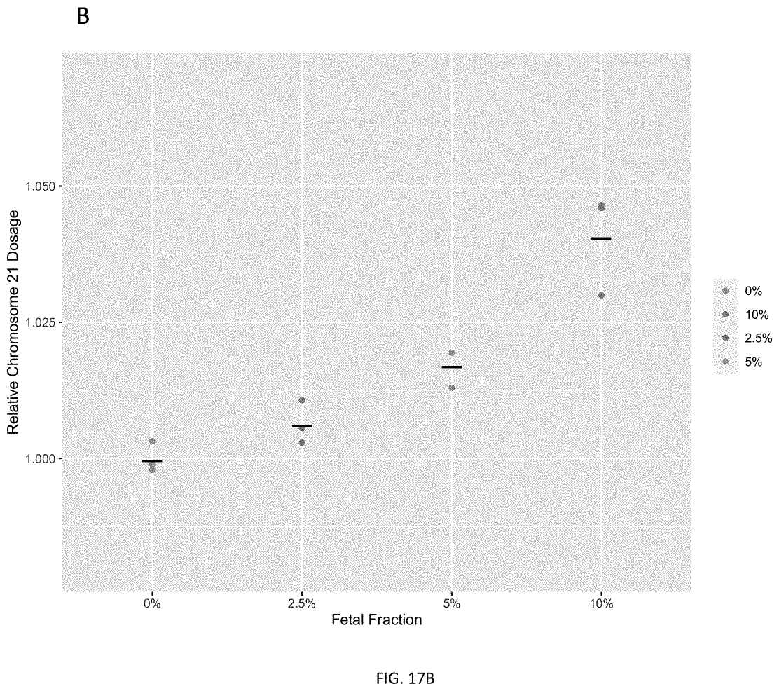

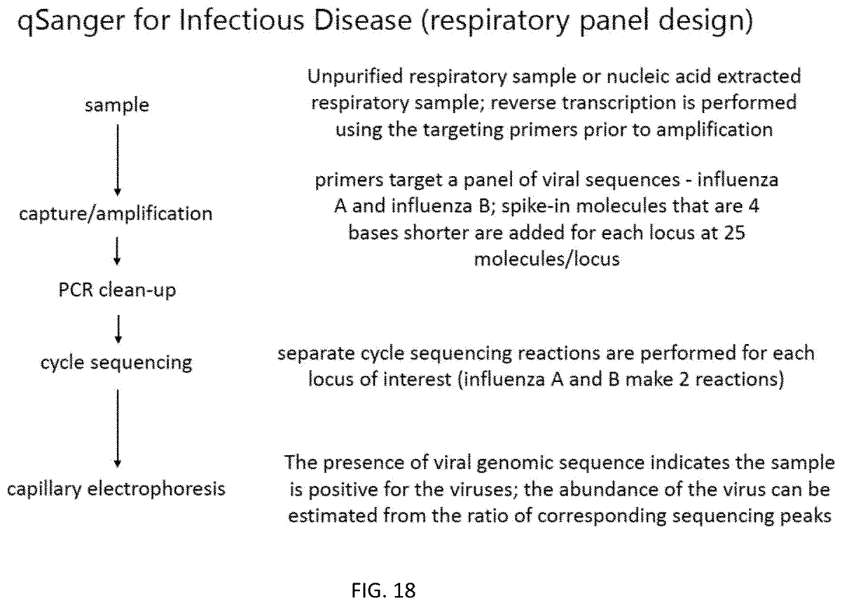

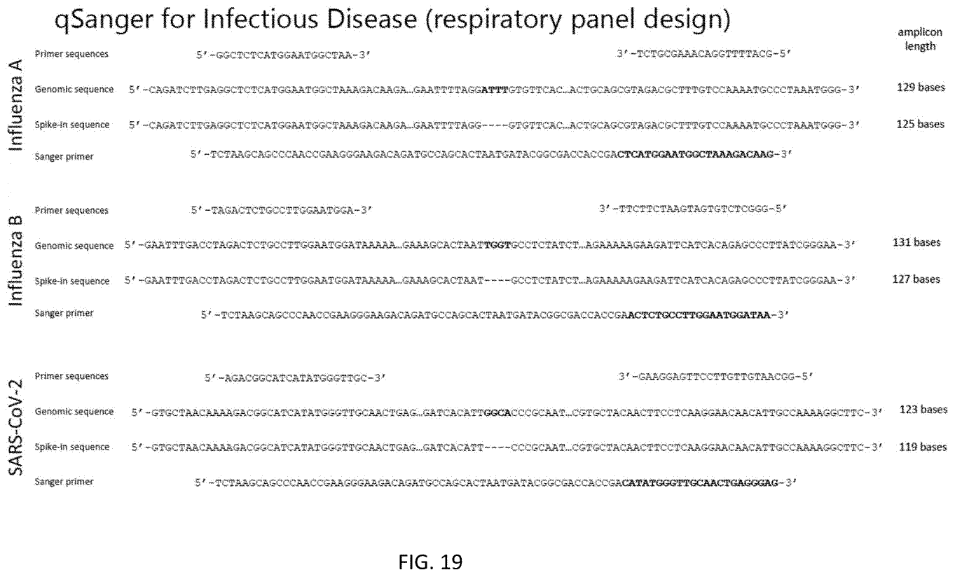

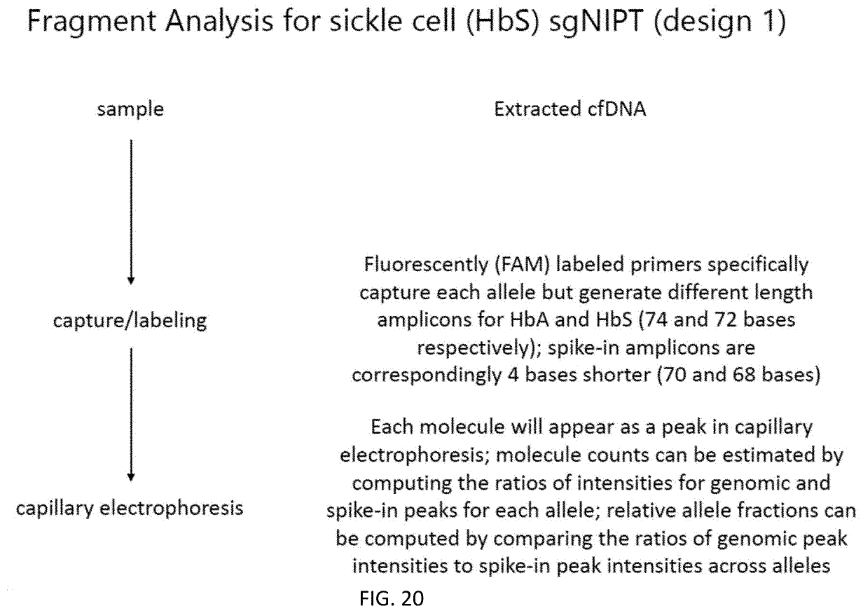

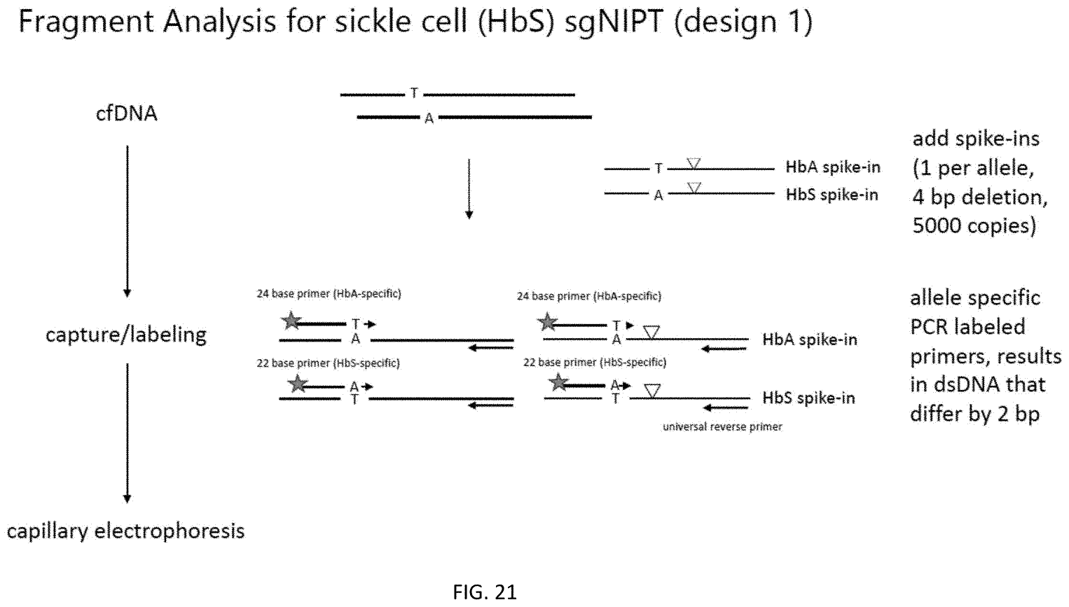

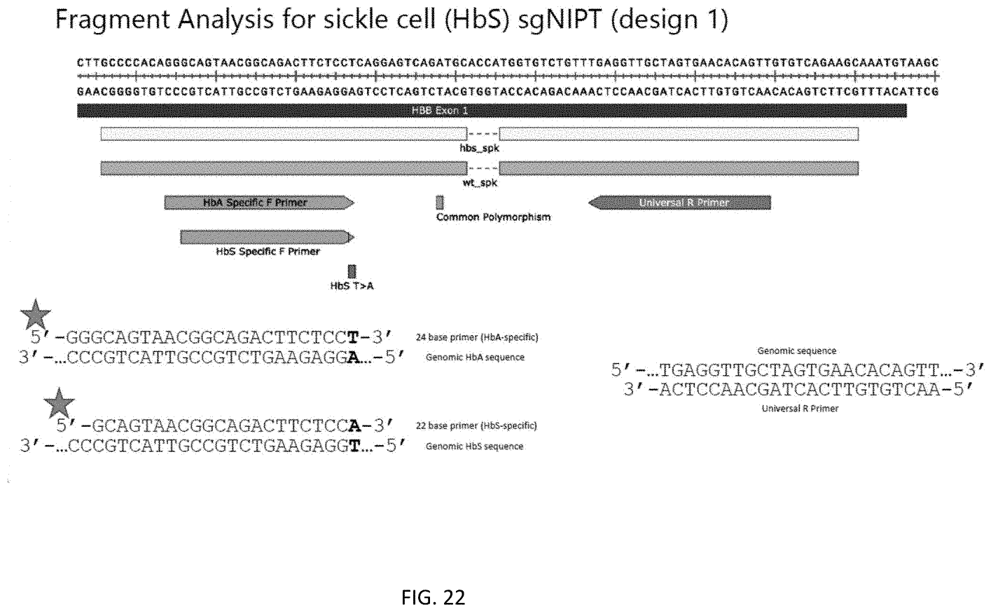

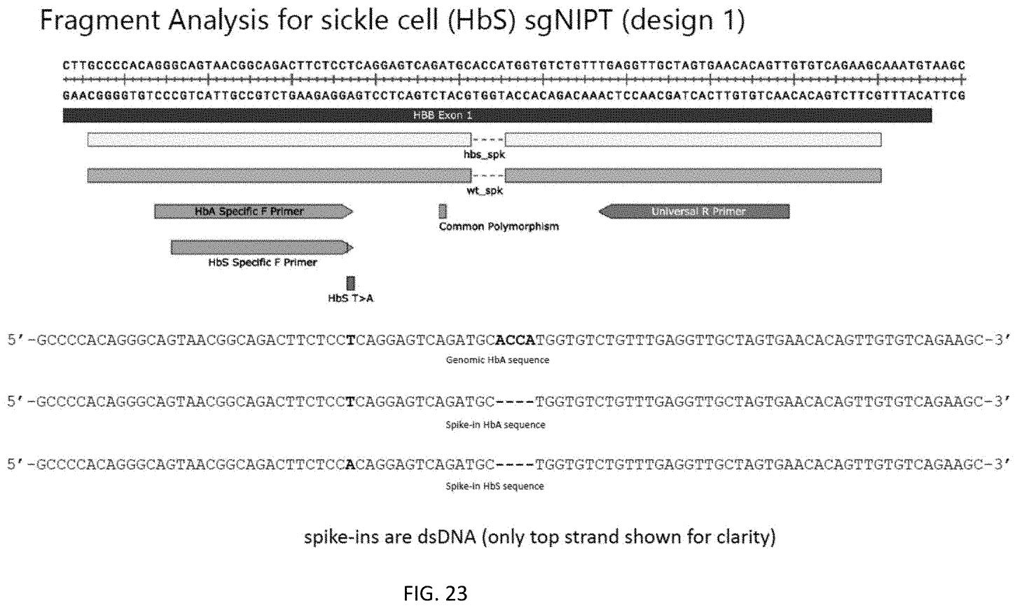

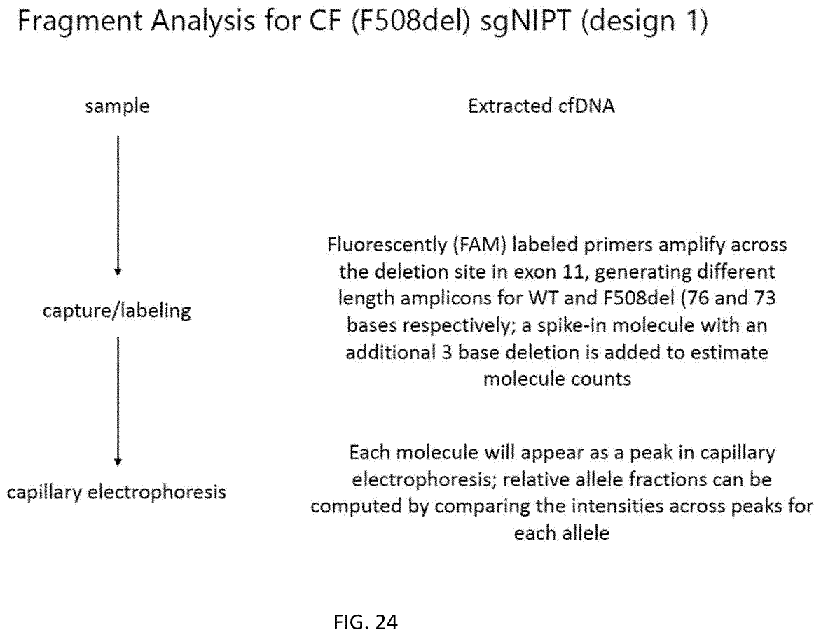

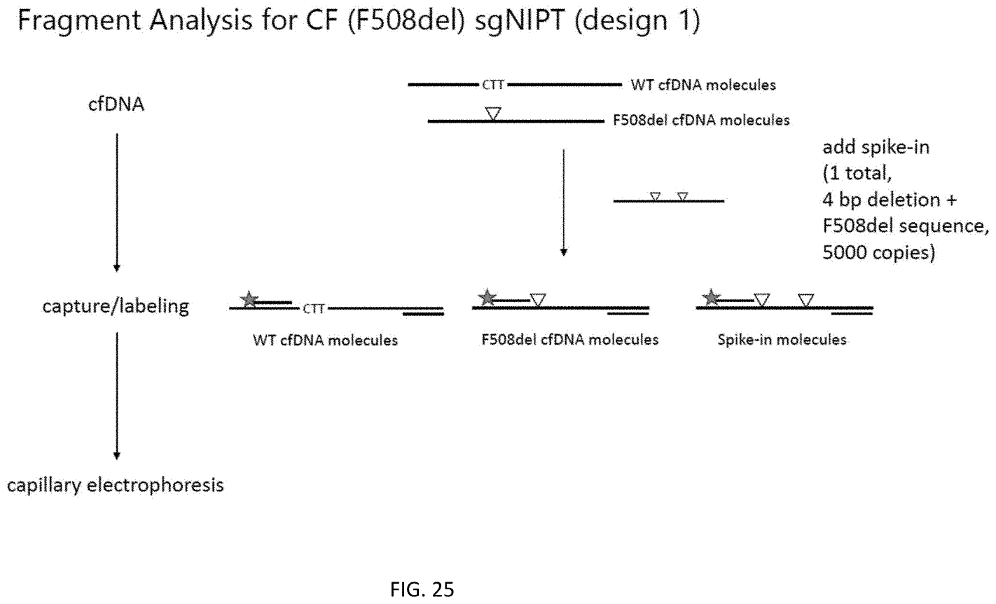

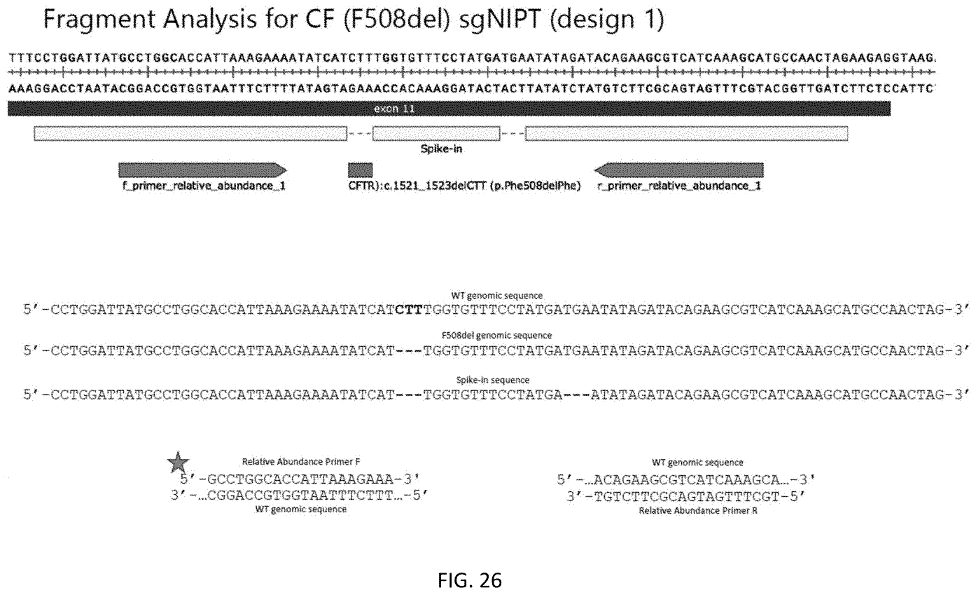

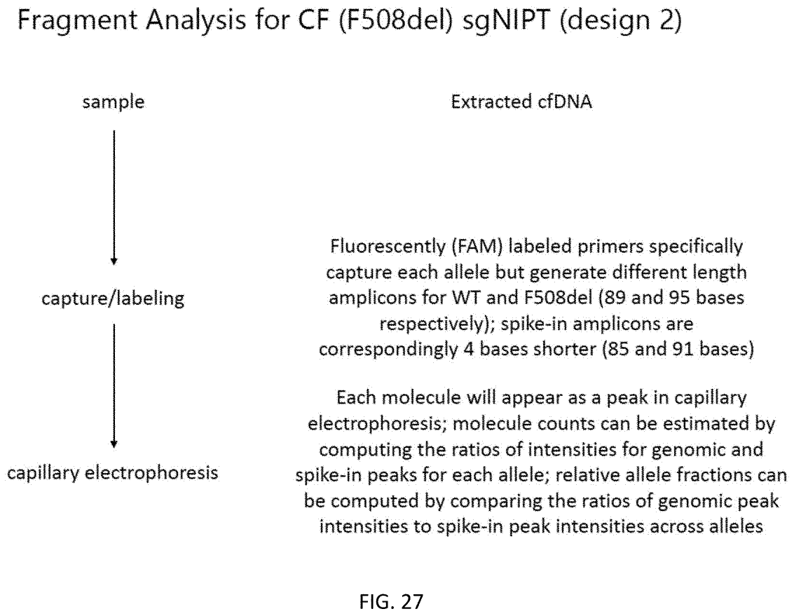

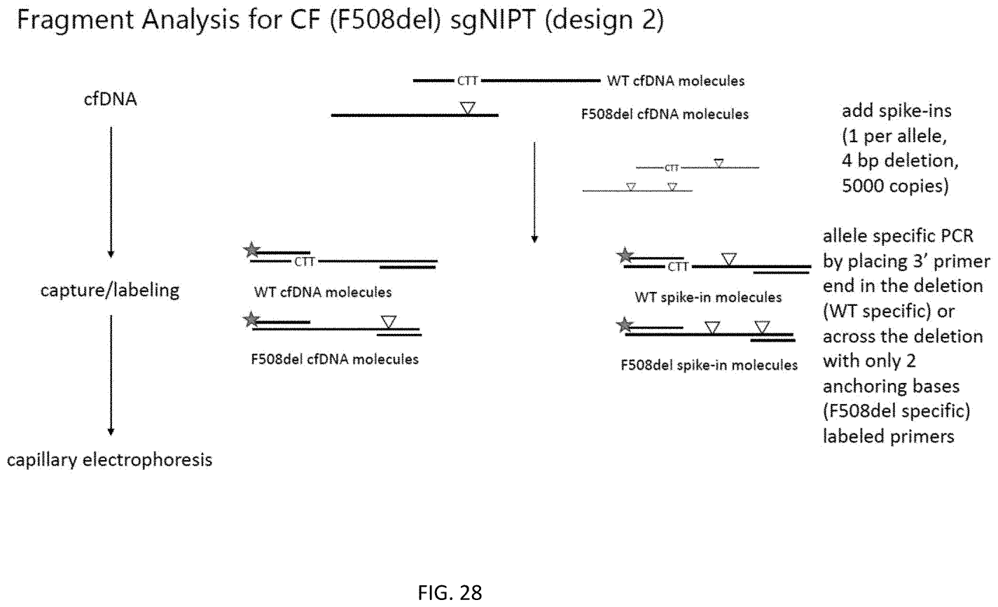

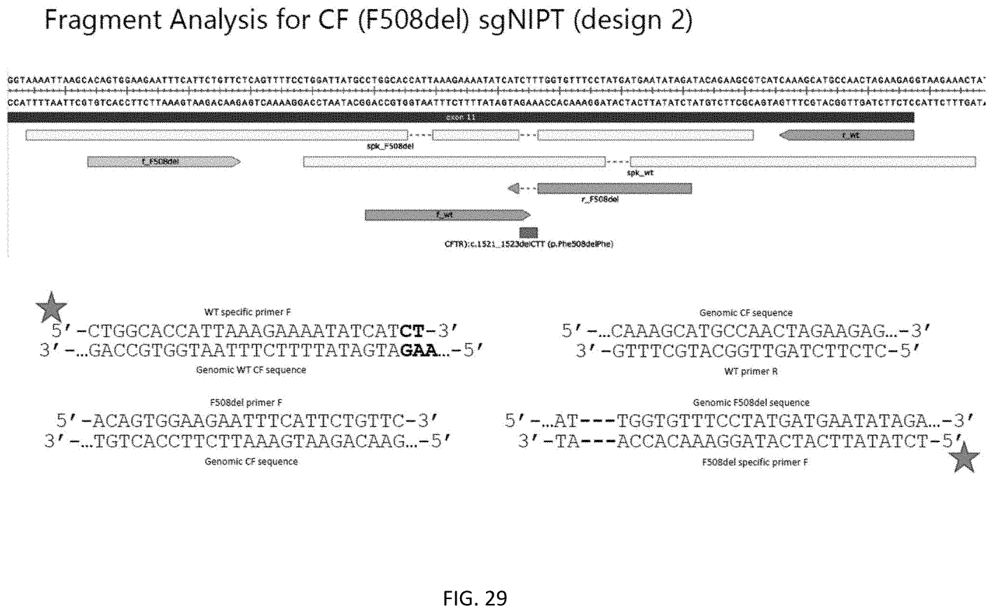

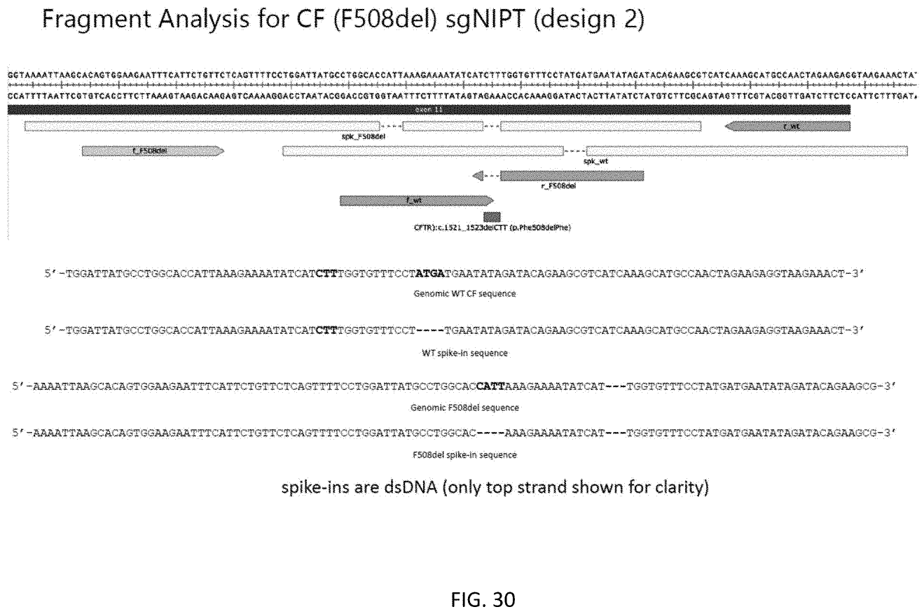

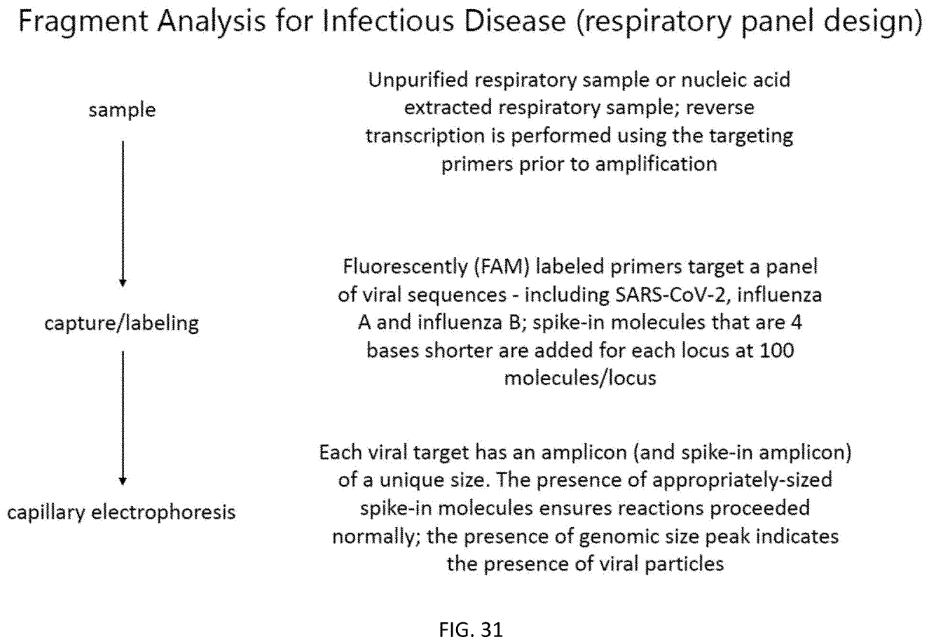

Applying the above methods to a positive sample. gDNA is sheared to mimic cfDNA and a positive sample (“fetal”) is mixed with “maternal” DNA (percentage of mixture is the x-axis of FIG. 17). There is a quantitative increase in the ratio as the percent of fetal DNA increases. A ratio of 1 is 100% euploid, and a ratio of 1.5 is 100% aneuploid. A is chromosome 18 and B is chromosome 21.

### General Fragment Analysis Protocol

Applying 1. Initial amplification/capture reaction (ex. multiplex PCR), to be used as input for labeling reaction (if desired)

1. For multiplex PCR:

1. Create typical master mix (use primer mix that target loci of interest, add appropriate enzyme)

1. Primer design must constrain lengths and molecular weights of resultant strands if labeling in multiplex; for convenient labeling, universal tailed sequences can be added

2. Include spike-in at functional concentration (ex. add spike-ins at the same per-locus concentration as the median expected sample); exact concentration is dependent on desired dynamic range of assay

3. PCR program specifics (ex. cycle count/annealing temperatures) depend on what is optimal for the designed primer set

2. Run labeling reaction (singleplex or multiplex PCR)

1. Input is either initial amplification/capture reaction product or sample+spike-in

2. Run typical PCR, use fluorescently labeled primer sets

1. Use fluorophores that are compatible with DNA Analyzer instrument (ex. Applied Biosystems 3730×1 DNA Analyzer)

2. Synthesize primers such that fluorophore is conjugated to only one end of resultant amplified product (fluorophore is incorporated at the 5′ end)

3. PCR program specifics (ex. cycle count/annealing temperatures) depend on what is optimal for the designed primer set

3. Inject using capillary electrophoresis (standard fragment analysis procedure)

1. Prepare labeling reaction product for injection

1. Dilute in formamide to an appropriate concentration (dilution amount determined empirically; dependent on labeling reaction yield)

1. Formamide is required to denature the DNA

2. Samples can additionally be heat denatured to ensure single-stranded product for injection

3. Include size standard in dilution, if desired (for calibration/quality control)

2. Add diluted sample to PCR plate type an appropriate volume (both must be compatible with the DNA Analyzer instrument) for injection

2. Inject on DNA Analyzer instrument (run capillary electrophoresis)

1. Injection conditions (injection time, voltage, run time, etc.) are dependent on sample details (ex. length of fragments, concentration) and instrument configuration (polymer type, capillary length)

2. Run replicate injections, if desired, to decrease measurement noise

### EQUIVALENTS AND INCORPORATION BY REFERENCE

While the (1) disclosure has been particularly shown and described with reference to a preferred embodiment and various alternate embodiments, it will be understood by persons skilled in the relevant art that various changes in form and details can be made therein without departing from the spirit and scope of the disclosure.

All referenced issued patents and patent applications cited within the body of the instant specification are hereby incorporated by reference in their entirety, for all purposes.

What is claimed is:

1. A method of determining the presence or absence of an aneuploidy, the method comprising:
mixing a DNA sample of a subject and a plurality of spike-in molecules to create a mixture, each of the plurality of spike-in molecules associated with a chromosome of a set of chromosomes, wherein each of the plurality of spike-in molecules comprises:
a target region having a first nucleotide sequence with sequence similarity to a target sequence region of the respective chromosome,
a variation region having a second nucleotide sequence with sequence dissimilarity to a sequence region of the respective chromosome, and
co-amplifying the mixture with one or more chromosome-specific primers to create a co-amplified mixture;
labeling the co-amplified mixture by chromosome with fluorescently labeled primers;
receiving peak data from the co-amplified mixture, the peak data including, for each chromosome of the set of chromosomes, genomic peak intensities of the DNA sample and spike-in peak intensities of the spike-in molecules associated with the respective chromosome;
for each chromosome, computing a ratio between the respective genomic peak intensity and the respective spike-in peak intensity;
determining the presence or absence of the aneuploidy based on the computed ratios.

2. The method of claim 1, wherein the one or more chromosome-specific primers includes a set of chromosome-specific primers, each chromosome-specific primer in the set configured to capture a respective chromosome with a tail of a discrete length of a set of discrete lengths.

3. The method of claim 2, wherein computing, for each chromosome, the ratio between the respective genomic peak intensity and the respective spike-in peak intensity comprises:
computing, for each discrete length of the set of discrete lengths, a ratio between the respective genomic peak intensity and the spike-in peak intensity;
aggregating of the computed ratios across each discrete length of the set of discrete lengths.

4. The method of claim 2, wherein computing, for each chromosome, the ratio between the respective genomic peak and the respective spike-in peak intensity comprises:
aggregating the genomic peak intensities across each discrete length of the set of discrete lengths;
aggregating the spike-in peak intensities across each discrete length of the set of discrete lengths;
computing a ratio between the aggregated genomic peak intensity and the aggregated spike-in peak intensity.

5. The method of claim 1, wherein the variation region includes an insertion of base pairs with a length of: one base pair, two base pairs, three base pairs, four base pairs, five base pairs, six base pairs, seven base pairs, eight base pairs, nine base pairs, ten base pairs, eleven base pairs, twelve base pairs, thirteen base pairs, fourteen base pairs, fifteen base pairs, sixteen base pairs, seventeen base pairs, eighteen base pairs, nineteen base pairs, or twenty base pairs.

6. The method of claim 1, wherein the variation region includes a deletion of base pairs with a length of: one base pair, two base pairs, three base pairs, four base pairs, five base pairs, six base pairs, seven base pairs, eight base pairs, nine base pairs, ten base pairs, eleven base pairs, twelve base pairs, thirteen base pairs, fourteen base pairs, fifteen base pairs, sixteen base pairs, seventeen base pairs, eighteen base pairs, nineteen base pairs, or twenty base pairs.

7. The method of claim 1, wherein a location of a respective variation region of a spike-in molecule is in the center of a respective amplicon of the spike-in molecule.

8. The method of claim 1, wherein each of the one or more fluorescently labeled primers is associated with a color channel.

9. A method of determining the presence or absence of an aneuploidy, the method comprising:
for each chromosome in a set of chromosomes:
mixing a DNA sample of a subject and a spike-in molecule of a plurality of spike-in molecules to create a mixture, each of the plurality of spike-in molecules associated with the chromosome of the set of chromosomes, wherein each of the plurality of spike-in molecules comprises:
a target region having a first nucleotide sequence with sequence similarity to a target sequence region of the respective chromosome,
a variation region having a second nucleotide sequence with sequence dissimilarity to a sequence region of the respective chromosome,
co-amplifying the mixture with one or more primers of a set of primers to generate a co-amplified mixture, each primer configured to capture the respective chromosome and add a tail with a discrete length of a set of discrete lengths to an amplicon of the DNA sample and add a tail with the discrete length of the set of discrete lengths to an amplicon of the spike-in molecule;
labeling the co-amplified mixture with fluorescently labeled primers;
receiving peak data from the co-amplified mixture, the peak data including genomic peak intensities of the portion of the DNA sample for each discrete length of the set of discrete lengths and the spike-in peak intensities of the spike-in molecule for each discrete length of the set of discrete lengths;
for each respective discrete length, computing a discrete length-specific ratio between the respective genomic peak intensity and the spike-in peak intensity; and
aggregating the discrete length-specific ratios across each of the discrete lengths in the set of discrete lengths to generate a chromosome-specific ratio; and
determining the presence or absence of aneuploidy based on the computed chromosome-specific ratios.

10. The method of claim 9, wherein determining the presence or absence of an aneuploidy based on the computed chromosome-specific ratios comprises:
computing the ratio of a chromosome-specific ratio to each of the other chromosome-specific ratios;
in response to determining a computed ratio is greater than a threshold ratio, determining the presence of aneuploidy; and
in response to determining a computed ratio is less than a threshold ratio, determining the absence of aneuploidy.

11. The method of claim 9, wherein the variation region includes an insertion of base pairs with a length of: one base pair, two base pairs, three base pairs, four base pairs, or five base pairs.

12. The method of claim 9, wherein the variation region includes a deletion of base pairs with a length of: one base pair, two base pairs, three base pairs, four base pairs, or five base pairs.

13. The method of claim 9, wherein a location of a respective variation region of a spike-in molecule is in the center of a respective amplicon of the spike-in molecule.

14. A method of determining the presence or absence of a genetic disorder in a noninvasive prenatal test, the method comprising:
mixing a genomic sample of a subject and one or more spike-in molecules associated with the genetic disorder, each spike-in molecule associated with an allele of the genetic disorder, wherein the spike-in molecule comprises:
a target region having a first nucleotide sequence with sequence similarity to a target sequence region of the respective allele of the genetic disorder,
a variation region having a second nucleotide sequence with sequence dissimilarity to a sequence region of the respective allele of the genetic disorder,
co-amplifying the mixture with one or more fluorescently labeled primers to generate a co-amplified mixture, wherein each of the one or more fluorescently labeled primers captures a respective allele of the genetic disorder, and wherein each of the fluorescently labeled primers generates an amplicon of the allele with a discrete length;
receiving peak data from the co-amplified mixture, the peak data including, for each of the captured alleles, genomic peak intensities of the genomic sample and spike-in peak intensities of the spike-in molecules;
computing, for each of the captured alleles, a ratio of the genomic peak intensity and the spike-in peak intensity; and
determining the presence or absence of the genetic disorder based on a comparison of the computed ratios across each of the captured alleles.

15. The method of claim 14, wherein each of the captured alleles is associated with a color channel.

16. The method of claim 14, wherein an amplicon of a first allele of the captured alleles has a first length, an amplicon of a second allele of the captured alleles as a second length, and wherein the first length is shorter than the second length.

17. The method of claim 14, wherein the variation region includes an insertion of base pairs with a length of: one base pair, two base pairs, three base pairs, four base pairs, or five base pairs.

18. The method of claim 14, wherein the variation region includes a deletion of base pairs with a length of: one base pair, two base pairs, three base pairs, four base pairs, or five base pairs.

19. The method of claim 14, wherein a location of a respective variation region of a spike-in molecule is in the center of a respective amplicon of the spike-in molecule.

20. The method of claim 14, wherein the genetic disorder is sickle cell.

21. The method of claim 20, wherein a first spike-in molecule is associated with HbS allele, and wherein a second spike-in molecule is associated with HbA allele.

22. The method of claim 21, wherein computing the ratio for each of captured alleles comprises:
computing a first ratio of peak intensities, wherein the first ratio is the ratio of the genomic peak intensity of the HbS allele and the spike-in intensity of the first spike-in molecule;
computing a second ratio of peak intensities, wherein the second ratio is the ratio of the genomic peak intensity of the HbA allele and the spike-in intensity of the second spike-in molecule; and
wherein determining the presence or absence a genetic disorder comprises determining the presence or absence of sickle cell disease based on a comparison of the first ratio and the second ratio.

23. The method of claim 14, wherein the genetic disorder is cystic fibrosis.

24. The method of claim 23, wherein a first spike-in molecule is associated with WT allele, and wherein a second spike-in molecule is associated with F508del allele.

25. The method of claim 24, wherein computing the ratio for each of captured alleles comprises:
computing a first ratio of peak intensities, wherein the first ratio is the ratio of the genomic peak intensity of the WT allele and the spike-in intensity of the first spike-in molecule;
computing a second ratio of peak intensities, wherein the second ratio is the ratio of the genomic peak intensity of the F508del allele and the spike-in intensity of the second spike-in molecule; and
wherein determining the presence or absence a genetic disorder comprises determining the presence or absence of cystic fibrosis disease based on a comparison of the first ratio and the second ratio.

26. The method of claim 14, wherein each of the one or more fluorescently labeled primers is associated with a color channel.

27. A method of determining the presence or absence of a genetic disorder in a noninvasive prenatal test, the method comprising:
mixing a genomic sample of a subject and a spike-in molecule associated with an allele of the genetic disorder to create a mixture, wherein the spike-in molecule includes a spike-in sequence, wherein the spike-in sequence comprises:
a target region having a nucleotide sequence with sequence similarity to a target sequence region of the allele of the genetic disorder,
a variation region having a nucleotide sequence with sequence dissimilarity to a sequence region of the allele of the genetic disorder,
co-amplifying the mixture with one or more sets of allele-specific primers to generate a co-amplified mixture, each primer in a set of allele-specific primers configured to capture the respective allele and add a tail with a discrete length of a set of discrete lengths to an amplicon of the genomic sample and add a tail with the discrete length of the set of discrete lengths to an amplicon of the spike-in molecule, the amplicon of the genomic sample including the target sequence, the amplicon of the spike-in molecule including the spike-in sequence;
labeling the co-amplified mixture with fluorescently labeled primers;
receiving peak data from the co-amplified mixture, the peak data including for each discrete length of the set of discrete lengths, genomic peak intensities of the genomic sample and spike-in peak intensities of the spike-in molecule;
for each respective discrete length, computing a ratio between the respective genomic peak intensity and the spike-in peak intensity; and
determining the presence or absence of the genetic disorder based on the computed ratios.

28. The method of claim 27, wherein computing, for each allele, the ratio between the respective genomic peak intensity and the respective spike-in peak intensity comprises:
computing, for each discrete length of the set of discrete lengths, a ratio between the respective genomic peak intensity and the spike-in peak intensity;
aggregating of the computed ratios across each discrete length of the set of discrete lengths.

29. The method of claim 27, wherein computing, for each allele, the ratio between the respective genomic peak intensity and the respective spike-in peak intensity comprises:
aggregating the genomic peak intensities across each discrete length of the set of discrete lengths;
aggregating the spike-in peak intensities across each discrete length of the set of discrete lengths;
computing a ratio between the aggregated genomic peak intensity and the aggregated spike-in peak intensity.

30. The method of claim 27, wherein the variation region includes an insertion of base pairs with a length of: one base pair, two base pairs, three base pairs, four base pairs, or five base pairs.

31. The method of claim 27, wherein the variation region includes a deletion of base pairs with a length of: one base pair, two base pairs, three base pairs, four base pairs, or five base pairs.

32. The method of claim 27, wherein a location of a respective variation region of a spike-in molecule is in the center of a respective amplicon of the spike-in molecule.

33. The method of claim 27, wherein each of the one or more fluorescently labeled primers is associated with a different fluorophore.

34. A method of determining the presence or absence of an aneuploidy, the method comprising:
mixing a DNA sample of a subject and a plurality of spike-in molecules to create a mixture, each of the plurality of spike-in molecules associated with a chromosome of a set of chromosomes, wherein each of the plurality of spike-in molecules comprises:
a target region having a first nucleotide sequence with sequence similarity to a target sequence region of the respective chromosome,
a variation region having a second nucleotide sequence with sequence dissimilarity to a sequence region of the respective chromosome, and
co-amplifying the mixture with one or more chromosome-specific primers to create a co-amplified mixture, wherein the one or more chromosome-specific primers are fluorescently labeled primers;
receiving peak data from the co-amplified mixture, the peak data including, for each chromosome of the set of chromosomes, genomic peak intensities of the DNA sample and spike-in peak intensities of the spike-in molecules associated with the respective chromosome;
for each chromosome, computing a ratio between the respective genomic peak intensity and the respective spike-in peak intensity;
determining the presence or absence of the aneuploidy based on the computed ratios.

35. The method of claim 34, wherein the one or more chromosome-specific primers includes a set of chromosome-specific primers, each chromosome-specific primer in the set configured to capture a respective chromosome with a tail of a discrete length of a set of discrete lengths.

36. The method of claim 35, wherein computing, for each chromosome, the ratio between the respective genomic peak intensity and the respective spike-in peak intensity comprises:
computing, for each discrete length of the set of discrete lengths, a ratio between the respective genomic peak intensity and the spike-in peak intensity;
aggregating of the computed ratios across each discrete length of the set of discrete lengths.

37. The method of claim 35, wherein computing, for each chromosome, the ratio between the respective genomic peak and the respective spike-in peak intensity comprises:
aggregating the genomic peak intensities across each discrete length of the set of discrete lengths;
aggregating the spike-in peak intensities across each discrete length of the set of discrete lengths;
computing a ratio between the aggregated genomic peak intensity and the aggregated spike-in peak intensity.

38. The method of claim 34, wherein the variation region includes an insertion of base pairs with a length of: one base pair, two base pairs, three base pairs, four base pairs, or five base pairs.

39. The method of claim 34, wherein the variation region includes a deletion of base pairs with a length of: one base pair, two base pairs, three base pairs, four base pairs, or five base pairs.

40. The method of claim 34, wherein a location of a respective variation region of a spike-in molecule is in the center of a respective amplicon of the spike-in molecule.

41. A method comprising:
mixing a nucleic acid sample of a subject and a spike-in molecule associated with an allele to create a mixture, wherein the spike-in molecule includes a spike-in sequence, wherein the spike-in sequence comprises:
a target region having a nucleotide sequence with sequence similarity to a target sequence region of the allele,
a variation region having a nucleotide sequence with sequence dissimilarity to a sequence region of the allele,
co-amplifying the mixture with one or more sets of allele-specific primers to generate a co-amplified mixture, each primer in a set of allele-specific primers configured to capture the respective allele and add a tail with a discrete length of a set of discrete lengths to an amplicon of the genomic sample and add a tail with the discrete length of the set of discrete lengths to an amplicon of the spike-in molecule, the amplicon of the genomic sample including the target sequence, the amplicon of the spike-in molecule including the spike-in sequence;
labeling the co-amplified mixture with one or more fluorescently labeled primers;
receiving peak data from the co-amplified mixture, the peak data including for each discrete length of the set of discrete lengths, genomic peak intensities of the genomic sample and spike-in peak intensities of the spike-in molecule;
for each respective discrete length, computing a ratio between the respective genomic peak intensity and the spike-in peak intensity; and
determining the presence or absence of the allele based on the computed ratios.

42. The method of claim 41, wherein computing, for each allele, the ratio between the respective genomic peak intensity and the respective spike-in peak intensity comprises:
computing, for each discrete length of the set of discrete lengths, a ratio between the respective genomic peak intensity and the spike-in peak intensity;
aggregating of the computed ratios across each discrete length of the set of discrete lengths.

43. The method of claim 41, wherein computing, for each allele, the ratio between the respective genomic peak intensity and the respective spike-in peak intensity comprises:
aggregating the genomic peak intensities across each discrete length of the set of discrete lengths;
aggregating the spike-in peak intensities across each discrete length of the set of discrete lengths;
computing a ratio between the aggregated genomic peak intensity and the aggregated spike-in peak intensity.

44. The method of claim 41, wherein the variation region includes an insertion of base pairs with a length of: one base pair, two base pairs, three base pairs, four base pairs, or five base pairs.

45. The method of claim 41, wherein the variation region includes a deletion of base pairs with a length of: one base pair, two base pairs, three base pairs, four base pairs, or five base pairs.

46. The method of claim 41, wherein a location of a respective variation region of a spike-in molecule is in the center of a respective amplicon of the spike-in molecule.

47. The method of claim 41, wherein each of the one or more fluorescently labeled primers is associated with a different fluorophore.

48. A method of determining the presence or absence of an aneuploidy, the method comprising:
for each chromosome in a set of chromosomes:
mixing a DNA sample of a subject and a spike-in molecule of a plurality of spike-in molecules to create a mixture, each of the plurality of spike-in molecules associated with the chromosome of the set of chromosomes, wherein each of the plurality of spike-in molecules comprises:
a target region having a first nucleotide sequence with sequence similarity to a target sequence region of the respective chromosome,
a variation region having a second nucleotide sequence with sequence dissimilarity to a sequence region of the respective chromosome,
co-amplifying the mixture with one or more primers to generate a co-amplified mixture, each primer configured to capture a respective chromosome;
for each length of a set of discrete lengths, adding a tail with the discrete length to a subset of amplicons in the co-amplified mixture;
labeling the co-amplified mixture with one or more fluorescently labeled primers;
receiving peak data from the co-amplified mixture, the peak data including genomic peak intensities of the portion of the DNA sample for each discrete length of the set of discrete lengths and the spike-in peak intensities of the spike-in molecule for each discrete length of the set of discrete lengths;
for each respective discrete length, computing a discrete length-specific ratio between the respective genomic peak intensity and the spike-in peak intensity;
aggregating the discrete length-specific ratios across each of the discrete lengths in the set of discrete lengths to generate a chromosome-specific ratio; and
determining the presence or absence of aneuploidy based on the computed chromosome-specific ratios.

49. The method of claim 48, wherein determining the presence or absence of an aneuploidy based on the computed chromosome-specific ratios comprises:
computing the ratio of a chromosome-specific ratio to each of the other chromosome-specific ratios;
in response to determining a computed ratio is greater than a threshold ratio, determining the presence of aneuploidy; and
in response to determining a computed ratio is less than a threshold ratio, determining the absence of aneuploidy.

50. The method of claim 48, wherein the variation region includes an insertion of base pairs with a length of: one base pair, two base pairs, three base pairs, four base pairs, or five base pairs.

51. The method of claim 48, wherein the variation region includes a deletion of base pairs with a length of: one base pair, two base pairs, three base pairs, four base pairs, or five base pairs.

52. The method of claim 48, wherein a location of a respective variation region of a spike-in molecule is in the center of a respective amplicon of the spike-in molecule.

53. A method of determining the abundance of endogenous targets, the method comprising:
mixing a nucleic acid sample of a subject and a plurality of spike-in molecules to create a mixture, each of the plurality of spike-in molecules are associated with an endogenous target or targets, wherein each of the plurality of spike-in molecules further comprises:
a target region having a first nucleotide sequence with sequence similarity to a target sequence region;
a variation region having a nucleotide sequence with sequence dissimilarity to the target sequence; and
co-amplifying the mixture with target specific primers to create a co-amplified mixture;
labeling the co-amplified mixture by fluorescently labeled primers;
receiving peak data from the co-amplified mixture, the peak data including, for each target of the set of targets, peak intensities of the nucleic acid sample and spike-in peak intensities of the spike-in molecules associated with each respective target;
for each target, computing a ratio between the respective target peak intensity and the respective spike-in peak intensity;
determining the abundance of the target based on computed ratios.


SEQUENCE LISTING (46 sequences)

SEQ ID NO: 1 - DNA, 20 bp, Artificial Sequence
  Description of Artificial Sequence Synthetic primer
ggctctcatggaatggctaa

SEQ ID NO: 2 - DNA, 20 bp, Artificial Sequence
  Description of Artificial Sequence Synthetic primer
gcattttggacaaagcgtct

SEQ ID NO: 3 - DNA, 100 bp, Influenza A virus
cagatcttgaggctctcatggaatggctaaagacaagagaattttaggatttgtgttcacactgcagcgtagacgctttgtccaaaatgccctaaatggg

SEQ ID NO: 4 - DNA, 96 bp, Artificial Sequence
  Description of Artificial Sequence Synthetic oligonucleotide
cagatcttgaggctctcatggaatggctaaagacaagagaattttagggtgttcacactgcagcgtagacgctttgtccaaaatgccctaaatggg

SEQ ID NO: 5 - DNA, 83 bp, Artificial Sequence
  Description of Artificial Sequence Synthetic primer
tctaagcagcccaaccgaagggaagacagatgccagcactaatgatacggcgaccaccgactcatggaatggctaaagacaag

SEQ ID NO: 6 - DNA, 21 bp, Artificial Sequence
  Description of Artificial Sequence Synthetic primer
tagactctgccttggaatgga

SEQ ID NO: 7 - DNA, 22 bp, Artificial Sequence
  Description of Artificial Sequence Synthetic primer
gggctctgtgatgaatcttctt

SEQ ID NO: 8 - DNA, 100 bp, Influenza B virus
gaatttgacctagactctgccttggaatggataaaaagaaagcactaattggtgcctctatctagaaaaagaagattcatcacagagcccttatcgggaa

SEQ ID NO: 9 - DNA, 96 bp, Artificial Sequence
  Description of Artificial Sequence Synthetic oligonucleotide
gaatttgacctagactctgccttggaatggataaaaagaaagcactaatgcctctatctagaaaaagaagattcatcacagagcccttatcgggaa

SEQ ID NO: 10 - DNA, 81 bp, Artificial Sequence
  Description of Artificial Sequence Synthetic primer
tctaagcagcccaaccgaagggaagacagatgccagcactaatgatacggcgaccaccgaactctgccttggaatggataa

SEQ ID NO: 11 - DNA, 21 bp, Artificial Sequence
  Description of Artificial Sequence Synthetic primer
agacggcatcatatgggttgc

SEQ ID NO: 12 - DNA, 23 bp, Artificial Sequence
  Description of Artificial Sequence Synthetic primer
ggcaatgttgttccttgaggaag

SEQ ID NO: 13 - DNA, 103 bp, Severe acute respiratory syndrome coronavirus 2
gtgctaacaaaagacggcatcatatgggttgcaactgaggatcacattggcacccgcaatcgtgctacaacttcctcaaggaacaacattgccaaaaggcttc

SEQ ID NO: 14 - DNA, 99 bp, Artificial Sequence
  Description of Artificial Sequence Synthetic oligonucleotide
gtgctaacaaaagacggcatcatatgggttgcaactgaggatcacattcccgcaatcgtgctacaacttcctcaaggaacaacattgccaaaaggcttc

SEQ ID NO: 15 - DNA, 83 bp, Artificial Sequence
  Description of Artificial Sequence Synthetic primer
tctaagcagcccaaccgaagggaagacagatgccagcactaatgatacggcgaccaccgacatatgggttgcaactgagggag

SEQ ID NO: 16 - DNA, 108 bp, Homo sapiens
cttgccccacagggcagtaacggcagacttctcctcaggagtcagatgcaccatggtgtctgtttgaggttgctagtgaacacagttgtgtcagaagcaaatgtaagc

SEQ ID NO: 17 - DNA, 24 bp, Artificial Sequence
  Description of Artificial Sequence Synthetic primer
gggcagtaacggcagacttctcct

SEQ ID NO: 18 - DNA, 22 bp, Artificial Sequence
  Description of Artificial Sequence Synthetic primer
gcagtaacggcagacttctcca

SEQ ID NO: 19 - DNA, 24 bp, Homo sapiens
tggagaagtctgccgttactgccc

SEQ ID NO: 20 - DNA, 23 bp, Homo sapiens
tgaggttgctagtgaacacagtt

SEQ ID NO: 21 - DNA, 95 bp, Homo sapiens
gccccacagggcagtaacggcagacttctcctcaggagtcagatgcaccatggtgtctgtttgaggttgctagtgaacacagttgtgtcagaagc

SEQ ID NO: 22 - DNA, 91 bp, Artificial Sequence
  Description of Artificial Sequence Synthetic oligonucleotide
gccccacagggcagtaacggcagacttctcctcaggagtcagatgctggtgtctgtttgaggttgctagtgaacacagttgtgtcagaagc

SEQ ID NO: 23 - DNA, 91 bp, Artificial Sequence
  Description of Artificial Sequence Synthetic oligonucleotide
gccccacagggcagtaacggcagacttctccacaggagtcagatgctggtgtctgtttgaggttgctagtgaacacagttgtgtcagaagc

SEQ ID NO: 24 - DNA, 109 bp, Homo sapiens
tttcctggattatgcctggcaccattaaagaaaatatcatctttggtgtttcctatgatgaatatagatacagaagcgtcatcaaagcatgccaactagaagaggtaag

SEQ ID NO: 25 - DNA, 96 bp, Homo sapiens
cctggattatgcctggcaccattaaagaaaatatcatctttggtgtttcctatgatgaatatagatacagaagcgtcatcaaagcatgccaactag

SEQ ID NO: 26 - DNA, 93 bp, Artificial Sequence
  Description of Artificial Sequence Synthetic oligonucleotide
cctggattatgcctggcaccattaaagaaaatatcattggtgtttcctatgatgaatatagatacagaagcgtcatcaaagcatgccaactag

SEQ ID NO: 27 - DNA, 90 bp, Artificial Sequence
  Description of Artificial Sequence Synthetic oligonucleotide
cctggattatgcctggcaccattaaagaaaatatcattggtgtttcctatgaatatagatacagaagcgtcatcaaagcatgccaactag

SEQ ID NO: 28 - DNA, 20 bp, Artificial Sequence
  Description of Artificial Sequence Synthetic primer
gcctggcaccattaaagaaa

SEQ ID NO: 29 - DNA, 20 bp, Homo sapiens
acagaagcgtcatcaaagca

SEQ ID NO: 30 - DNA, 158 bp, Homo sapiens
ggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaacta

SEQ ID NO: 31 - DNA, 27 bp, Artificial Sequence
  Description of Artificial Sequence Synthetic primer
ctggcaccattaaagaaaatatcatct

SEQ ID NO: 32 - DNA, 28 bp, Homo sapiens
aagatgatattttctttaatggtgccag

SEQ ID NO: 33 - DNA, 25 bp, Artificial Sequence
  Description of Artificial Sequence Synthetic primer
acagtggaagaatttcattctgttc

SEQ ID NO: 34 - DNA, 22 bp, Homo sapiens
caaagcatgccaactagaagag

SEQ ID NO: 35 - DNA, 27 bp, Artificial Sequence
  Description of Artificial Sequence Synthetic oligonucleotide
attggtgtttcctatgatgaatataga

SEQ ID NO: 36 - DNA, 109 bp, Homo sapiens
tggattatgcctggcaccattaaagaaaatatcatctttggtgtttcctatgatgaatatagatacagaagcgtcatcaaagcatgccaactagaagaggtaagaaact

SEQ ID NO: 37 - DNA, 105 bp, Artificial Sequence
  Description of Artificial Sequence Synthetic polynucleotide
tggattatgcctggcaccattaaagaaaatatcatctttggtgtttccttgaatatagatacagaagcgtcatcaaagcatgccaactagaagaggtaagaaact

SEQ ID NO: 38 - DNA, 115 bp, Artificial Sequence
  Description of Artificial Sequence Synthetic polynucleotide
aaaattaagcacagtggaagaatttcattctgttctcagttttcctggattatgcctggcaccattaaagaaaatatcattggtgtttcctatgatgaatatagatacagaagcg

SEQ ID NO: 39 - DNA, 111 bp, Artificial Sequence
  Description of Artificial Sequence Synthetic polynucleotide
aaaattaagcacagtggaagaatttcattctgttctcagttttcctggattatgcctggcacaaagaaaatatcattggtgtttcctatgatgaatatagatacagaagcg

SEQ ID NO: 40 - DNA, 24 bp, Homo sapiens
aggagaagtctgccgttactgccc

SEQ ID NO: 41 - DNA, 23 bp, Artificial Sequence
  Description of Artificial Sequence Synthetic primer
aactgtgttcactagcaacctca

SEQ ID NO: 42 - DNA, 20 bp, Homo sapiens
tttctttaatggtgccaggc

SEQ ID NO: 43 - DNA, 20 bp, Artificial Sequence
  Description of Artificial Sequence Synthetic primer
tgctttgatgacgcttctgt

SEQ ID NO: 44 - DNA, 25 bp, Homo sapiens
gaacagaatgaaattcttccactgt

SEQ ID NO: 45 - DNA, 22 bp, Artificial Sequence
  Description of Artificial Sequence Synthetic primer
ctcttctagttggcatgctttg

SEQ ID NO: 46 - DNA, 27 bp, Artificial Sequence
  Description of Artificial Sequence Synthetic primer
tctatattcatcataggaaacaccaat